(19) United States
(12) Patent Application Publication
(10) Pub. No.: US 2022/0056111 A1
(43) Pub. Date: Feb. 24, 2022

(54) MONOCLONAL ANTIBODY AGAINST SPIKE S1 PROTEIN OF SARS-CoV-2 AND USE THEREOFOF

(71) Applicant: YUROGEN BIOSYSTEMS LLC, Worcester, MA (US)

(72) Inventors: Hai WU, Worcester, MA (US); Yang LOU, Worcester, MA (US)

(21) Appl. No.: 17/399,364

(22) Filed: Aug. 11, 2021

Related U.S. Application Data
(60) Provisional application No. 63/064,084, filed on Aug. 11, 2020.

(51) Int. Cl. C07K 16/10; C07K 19/00; G01N 33/543; C12N 9/08

(52) U.S. Cl. CPC C07K 16/10; C07K 19/00; G01N 33/543; G01N 2333/165; C07K 2317/565; C07K 2317/24; C12Y 111/01007; C12N 9/0065

(57) ABSTRACT

The disclosure provides rabbit monoclonal antibodies against the spike S1 protein of SARS-CoV-2 and uses thereof. The antibody comprises: a V_{H} CDR1 selected from the group consisting of SEQ ID NO: 1-7; a V_{H} CDR2 selected from the group consisting SEQ ID NO: 8-14; a V_{H} CDR3 selected from the group consisting of SEQ ID NO: 15-21; a V_{L} CDR1 selected from the group consisting SEQ IDN NO: 22-28; a V_{L} CDR2 selected from the group consisting of SEQ ID NO: 29-35; and a V_{L} CDR3 selected from the group consisting of SEQ ID NO: 36-42. The antibodies can be used for a rapid test or screening of SARS-CoV-2 infection. The antibodies can also be used for treating SARS-CoV-2 infection.

### FIELD

The disclosure relates to monoclonal antibodies (mAbs) against SARS-CoV-2. Specifically, the disclosure relates to rabbit monoclonal antibodies against the SARS-CoV-2 Spike S1 protein and use thereof.

### BACKGROUND

The SARS-CoV-2 refers to the severe acute respiratory syndrome coronavirus 2 (SARS-CoV-2) initially identified in December 2019 that causes coronavirus disease 2019 (COVID-19). SARS-CoV-2 uses the envelope spike (S) glycoprotein to mediate host cell entry. The S glycoprotein includes a subunit S1, namely Spike S1 protein or S1. The S1 facilitates SARS-CoV-2 to attach to a cell surface receptor, angiotensin-converting enzyme 2 (ACE2), via its receptor-binding domain (RBD). Disruption of the RBD-ACE2 interaction can block SARS-CoV-2 cell entry, thus offering potential therapeutic applications.

### SUMMARY

Embodiments provide monoclonal antibodies against the SARS-CoV-2 S1 and use thereof. In an embodiment, the monoclonal antibody comprises a V_{H} CDR1 having the amino acid sequence of SEQ ID NO: 1, a V_{H} CDR2 having the amino acid sequence of SEQ ID NO: 8, a V_{H} CDR3 having the amino acid sequence of SEQ ID NO: 15, a V_{L} CDR1 having the amino acid sequence of SEQ ID NO: 22, a V_{L} CDR2 having the amino acid sequence of SEQ ID NO: 29, and a V_{L} CDR3 having the amino acid sequence of SEQ ID NO: 36. In an embodiment, the monoclonal antibody comprises a V_{H} CDR1 having the amino acid sequence of SEQ ID NO: 2, a V_{H} CDR2 having the amino acid sequence of SEQ ID NO: 9, V_{H} CDR3 having the amino acid sequence of SEQ ID NO: 16, a V_{L} CDR1 having the amino acid sequence of SEQ ID NO: 23, a V_{L} CDR2 having the amino acid sequence of SEQ ID NO: 30, and a V_{L} CDR3 having the amino acid sequence of SEQ ID NO: 37. In an embodiment, the monoclonal antibody comprises a V_{H} CDR1 having the amino acid sequence of SEQ ID NO: 3, a V_{H} CDR2 having the amino acid sequence of SEQ ID NO: 10, V_{H} CDR3 having the amino acid sequence of SEQ ID NO: 17, a V_{L} CDR1 having the amino acid sequence of SEQ ID NO: 24, a V_{L} CDR2 having the amino acid sequence of SEQ ID NO: 31, and a V_{L} CDR3 having the amino acid sequence of SEQ ID NO: 38. In an embodiment, the monoclonal antibody comprises a V_{H} CDR1 having the amino acid sequence of SEQ ID NO: 4, a V_{H} CDR2 having the amino acid sequence of SEQ ID NO: 11, a V_{H} CDR3 having the amino acid sequence of SEQ ID NO: 18, a V_{L} CDR1 having the amino acid sequence of SEQ ID NO: 25, a V_{L} CDR2 having the amino acid sequence of SEQ ID NO: 32, and a V_{L} CDR3 having the amino acid sequence of SEQ ID NO: 39. In an embodiment, the monoclonal antibody comprises a V_{H} CDR1 having the amino acid sequence of SEQ ID NO: 5, a V_{H} CDR2 having the amino acid sequence of SEQ ID NO: 12, a V_{H} CDR3 having the amino acid sequence of SEQ ID NO: 19, a V_{L} CDR1 having the amino acid sequence of SEQ ID NO: 26, a V_{L} CDR2 having the amino acid sequence of SEQ ID NO: 33, and a V_{L} CDR3 having the amino acid sequence of SEQ ID NO: 40. In an embodiment, the monoclonal antibody comprises a V_{H} CDR1 having the amino acid sequence of SEQ ID NO: 6, a V_{H} CDR2 having the amino acid sequence of SEQ ID NO: 13, a V_{H} CDR3 having the amino acid sequence of SEQ ID NO: 20, a V_{L} CDR1 having the amino acid sequence of SEQ ID NO: 27, a V_{L} CDR2 having the amino acid sequence of SEQ ID NO: 34, and a V_{L} CDR3 having the amino acid sequence of SEQ ID NO: 41. In an embodiment, the monoclonal antibody comprises a V_{H} CDR1 having the amino acid sequence of SEQ ID NO: 7, a V_{H} CDR2 having the amino acid sequence of SEQ ID NO: 14, a V_{H} CDR3 having the amino acid sequence of SEQ ID NO: 21, a V_{L} CDR1 having the amino acid sequence of SEQ ID NO: 28, a V_{L} CDR2 having the amino acid sequence of SEQ ID NO: 35, and a V_{L} CDR3 having the amino acid sequence of SEQ ID NO: 42.

In an embodiment, the monoclonal antibody of claim 1 comprises a V_{H} and a V_{L}. In an embodiment, the V_{H} comprises the amino acid sequence of SEQ ID NO: 43, and the V_{L} comprises the amino acid sequence of SEQ ID NO: 50. In an embodiment, the V_{H} comprises the amino acid sequence of SEQ ID NO: 44, and the V_{L} comprises the amino acid sequence of SEQ ID NO: 51. In an embodiment, the V_{H} comprises the amino acid sequence of SEQ ID NO: 45, and the V_{L} comprises the amino acid sequence of SEQ ID NO: 52. In an embodiment, the V_{H} comprises the amino acid sequence of SEQ ID NO: 46, and the V_{L} comprises the amino acid sequence of SEQ ID NO: 53. In an embodiment, the V_{H} comprises the amino acid sequence of SEQ ID NO: 47, and the V_{L} comprises the amino acid sequence of SEQ ID NO: 54. In an embodiment, the V_{H} comprises the amino acid sequence of SEQ ID NO: 48, and the V_{L} comprises the amino acid sequence of SEQ ID NO: 55. In an embodiment, the V_{H} comprises the amino acid sequence of SEQ ID NO: 49, and the V_{L} comprising the amino acid sequence of SEQ ID NO: 56.

In an embodiment, the monoclonal antibody further comprises a covalently or non-covalently attached conjugate. In an embodiment, the conjugate includes an enzyme, a fluorescence protein, a fluorophore, biotin, or streptavidin. In an embodiment, the enzyme includes HRP. In an embodiment, the monoclonal antibody is a humanized or chimeric antibody for potential therapeutic applications.

The disclosure herein also provides a kit for detecting SARS-CoV-2 or a spike S1 of SARS-CoV-2, and the kit comprises the monoclonal antibody.

Embodiments further provide a method for a rapid test of SARS-CoV-2 infection or fast screening of SARS-CoV-2 carriers. In an embodiment, the method can include mixing a sample with the rabbit mAb against the SARS-CoV-2 S1. In an embodiment, the method is based on a direct ELISA. In an embodiment, the method is based on a capture ELISA. In an embodiment, the method is based on a sandwich ELISA. In an embodiment, the method further includes: adding a secondary antibody comprising a conjugate for detection.

### BRIEF DESCRIPTION OF DRAWINGS

FIG. 1 illustrates a structure of a rabbit mAb against the SARS-CoV-2 S1, in accordance with an embodiment.

FIG. 2A illustrates the sequence alignment of CDR1s of heavy chains of the rabbit mAb against the SARS-CoV-2 S1, in accordance with embodiments, including 1A3, 1D2, 1H1, 5E1, 7G5, 9A5, and 9H1.

FIG. 2B illustrates the sequence alignment of CDR2s of heavy chains of the rabbit mAb against the SARS-CoV-2 S1, in accordance with embodiments, including in accordance with embodiments, including 1A3, 1D2, 1H1, 5E1, 7G5, 9A5, and 9H1.

FIG. 2C illustrates the sequence alignment of CDR3s of heavy chains of the rabbit mAb against the SARS-CoV-2 S1, in accordance with embodiments, including 1A3, 1D2, 1H1, 5E1, 7G5, 9A5, and 9H1.

FIG. 3A illustrates the sequence alignment of CDR1s of light chains of the rabbit mAb against the SARS-CoV-2 S1, in accordance with embodiments, including 1A3, 1D2, 1H1, 5E1, 7G5, 9A5, and 9H1.

FIG. 3B illustrates the sequence alignment of CDR2s of light chains of the rabbit mAb against the SARS-CoV-2 S1, in accordance with embodiments, including 1A3, 1D2, 1H1, 5E1, 7G5, 9A5, and 9H1.

FIG. 3C illustrates the sequence alignment of CDR3s of light chains of the rabbit mAb against the SARS-CoV-2 S1, in accordance with embodiments, including 1A3, 1D2, 1H1, 5E1, 7G5, 9A5, and 9H1.

FIG. 4 illustrates the sequence alignment of variable regions of heavy chains of the rabbit mAb against the SARS-CoV-2 S1, in accordance with embodiments, including 1A3, 1D2, 1H1, 5E1, 7G5, 9A5, and 9H1.

FIG. 5 illustrates the sequence alignment of variable regions of light chains of the rabbit mAb against the SARS-CoV-2 S1, in accordance with embodiments, including 1A3, 1D2, 1H1, 5E1, 7G5, 9A5, and 9H1.

FIG. 6 illustrates the sequence alignment of heavy chains of Fab fragments of the rabbit mAb against the SARS-CoV-2 S1, in accordance with embodiments, including 1A3, 1D2, 1H1, 5E1, 7G5, 9A5, and 9H1.

FIG. 7 illustrates the sequence alignment of light chains of Fab fragments of the rabbit mAb against the SARS-CoV-2 S1, in accordance with embodiments, including 1A3, 1D2, 1H1, 5E1, 7G5, 9A5, and 9H1.

FIG. 8 A shows direct antigen ELISA results of 1A3, 1D2, 1H1, 5E1, 7G5, 9A5, and 9H1 for detecting the SARS-CoV-2 S1, in accordance with an embodiment.

FIG. 8 B shows direct antigen ELISA results of 1A3, 1D2, 1H1, 5E1, 7G5, 9A5, and 9H1 for detecting RBD (receptor binding domain), in accordance with an embodiment.

FIG. 9 A shows capture ELISA results of 1A3, 1D2, 1H1, 5E1, 7G5, 9A5, and 9H1 for detecting the SARS-CoV-2 S1, in accordance with an embodiment.

FIG. 9 B shows capture ELISA results of 1A3, 1D2, 1H1, 5E1, 7G5, 9A5, and 9H1 for detecting RBD, in accordance with an embodiment.

FIG. 10 shows specificity of 1A3, 1D2, 1H1, 5E1, 7G5, 9H1, and 9A5 toward SARS-CoV-2 (SARS-CoV-2) S1, SARS S1 and S2 protein, MERS-CoV Spike protein, HKU1 S1 and S2 protein, HcoV-NL63 S protein, HcoV-OC43 S protein, and HcoV-229E S protein, in accordance with an embodiment.

FIG. 11 shows results of neutralizing capability of 1H1, 9H1, 5E1, and 7G5 in pseudovirus infection assay, in accordance with an embodiment.

FIG. 12 shows results of blocking assay to quantify the strength of blocking activity against ACE2-S1 binding of 1H1, 9H1, 5E1, and 7G5, in accordance with an embodiment.

FIG. 13 A shows results of sandwich ELISA using 1H1 as the capture antibody and 1A3 as the detection antibody, in accordance with an embodiment.

FIG. 13 B shows results of sandwich ELISA using 5E1 as the capture antibody and 1D2 as the detection antibody, in accordance with an embodiment.

FIG. 13 C shows results of sandwich ELISA using 7G5 as the capture antibody and 1H1 as the detection antibody, in accordance with an embodiment.

FIG. 13 D shows results of sandwich ELISA using 9A5 as the capture antibody and 5E1 as the detection antibody, in accordance with an embodiment.

FIG. 13 E shows results of sandwich ELISA using 9A5 as the capture antibody and 9H1 as the detection antibody, in accordance with an embodiment.

FIG. 13 F shows results of sandwich ELISA using 9H1 as the capture antibody and 9A5 as the detection antibody, in accordance with an embodiment.

### DETAILED DESCRIPTION

The disclosure generally relates to antibodies against SARS-CoV-2. Specifically, the disclosure relates to rabbit monoclonal antibodies (mAbs) against the SARS-CoV-2 S1 and use thereof.

The term “antibody” herein can be used in the broadest sense and encompasses various antibody structures, including but not limited to a Y-shaped antibody, namely full-length antibody, an antigen-binding portion of the Y-shaped antibody, and a genetic or chemical modification thereof. The antigen-binding portion refers to one or more portions or fragments of the Y-shaped antibody and can retain the ability of the antibody to bind to the SARS-CoV-2 S1 specifically.

The term “monoclonal antibody” (mAb) refers to an antibody having a substantially homogeneous population. The individual antibodies of the population are substantially identical, except for possible naturally occurring mutations that may be present in minor amounts. A monoclonal antibody can display a single binding specificity and affinity for a particular epitope on an antigen. In contrast to polyclonal antibodies that typically include different antibodies directed against different epitopes, each monoclonal antibody can target the same or substantially identical epitope on the antigen. The modifier “monoclonal” indicates the character of the antibody as being obtained from a substantially homogeneous population of antibodies, and is not to be construed as requiring the production of the antibody by any particular method. The antibody can be made by various methods, including but limited to, for example, hybridoma method, recombinant DNA methods, phage antibody libraries, and the like.

The terms “mAb against the SARS-CoV-2 S1,” “mAb against the spike S1 protein of SARS-CoV-2,” and “mAb against the S1” are used interchangeably and refer to monoclonal antibodies capable of binding to the spike S1 protein of SARS-CoV-2 with sufficient affinity so that the antibodies are useful as a detecting, diagnostic, and/or therapeutic agent in targeting SARS-CoV-2. The term “affinity” refers to the strength of the total of non-covalent intermolecular interactions between a single binding site of a molecule (e.g., an antibody) and its binding partner (e.g., an antigen). The intermolecular interactions can include hydrogen bonding, electrostatic interactions, hydrophobic, and Van der Waals forces.

The modifier “rabbit” in the term “rabbit antibody” or “rabbit mAb against the SARS-CoV-2 S1” or the like indicates the complementarity-determining regions (CDRs) of the antibody are derived from rabbit germline immunoglobulin sequences. In an embodiment, the rabbit mAb against the SARS-CoV-2 S1 may include antibodies whose CDRs and FRs are derived from rabbit germline immunoglobulin sequences. In an embodiment, the rabbit antibody or rabbit mAb against the spike S1 of SARS-CoV-2 can encompass antibodies whose CDRs are derived from rabbit germline immunoglobulin sequences. In an embodiment, the rabbit mAb against the SARS-CoV-2 S1 may encompass antibodies whose CDRs are derived from rabbit germline immunoglobulin sequences and whose framework regions (FRs) are derived from germline immunoglobulin sequences of another mammalian species, such as mouse or human. The term “rabbit mAb against the SARS-CoV-2 S1” may also encompass antibodies containing amino acid residues not encoded by rabbit germline immunoglobulin sequences, e.g., mutations introduced by random or site-specific mutagenesis in vitro or by somatic mutation in vivo. However, the term “rabbit mAb against the SARS-CoV-2 S1” is not intended to include antibodies whose CDRs are derived from the germline of another mammalian species, such as a mouse.

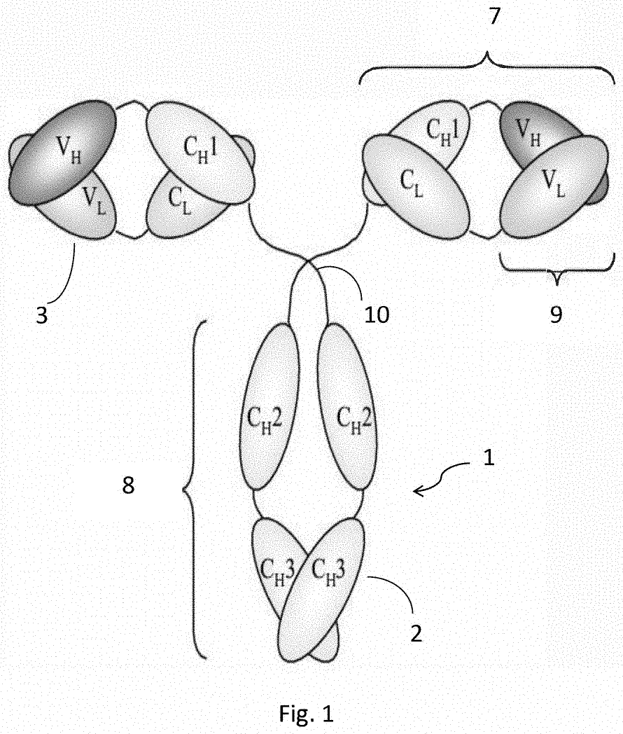

In an embodiment, the rabbit mAb against the SARS-CoV-2 S1 can be a Y-shaped antibody. Referring to FIG. 1, FIG. 1 illustrates a Y-shaped structure of a rabbit mAb against the SARS-CoV-2 S1 in accordance with an embodiment. In an embodiment, the rabbit mAb against the SARS-CoV-2 S1 can include two pairs of heavy chain 2 and light chain 3. The heavy chain 2 can include one variable region (V_{H}) and one or more constant regions (C_{HS}). In an embodiment, the heavy chain 2 can include one V_{H} and three C_{HS}, namely C_{H}1, C_{H}2, and C_{H}3. The V_{H} is closer to the N-terminus of the heavy chain as compared to the three C_{HS}. The V_{H} can exhibit higher variability in the amino acid sequence as compared to the C_{HS}. The V_{H} can differ between different antibodies and can be specific to each antibody. The amino acid sequences of the C_{HS} can be identical across all antibodies of the same isotype (class) but differ between isotypes. The term “isotype” refers to the antibody class (e.g., IgG) encoded by the heavy-chain constant-region genes. Mammalian antibodies can include five types of heavy chains: γ, δ, α, μ, and ε. They define classes of antibodies: IgG, IgD, IgA, IgM, and IgE, respectively.

The light chain 3 can be a small polypeptide subunit relative to the heavy chain 2. The light chain 3 can include one variable region (V_{L}) and one constant (C_{L}) region. The V_{L} is generally is the N-terminus portion of the light chain 3 and exhibits higher variability in amino acid sequence than the C_{L}. The V_{L} can differ between different antibodies and be specific to each antibody in amino acid sequence.

In an embodiment, the variable regions, V_{H} and V_{L}, are responsible for recognizing and binding the S1. In an embodiment, C_{HS} and C_{L} do not directly contact residues of the spike S1 protein.

The two pairs of heavy chain 2 and light chain 3 can form a Y-shaped structure that includes two Fab (Fragment antigen-binding) fragments 7, one Fc (Fragment crystallizable) fragment 8, and hinge regions 10. The two Fab fragments 7 look like the two arms of the “Y”, and the Fc fragment 8 looks like the base of the “Y.” The hinge regions 10 connect the Fc fragment 8 with the two Fab fragments 7.

Each of the Fab fragments 7 can comprise the V_{H} and C_{H}1 from the heavy chain 2 and the V_{L} and C_{L} from the light chain 3. The Fab fragment 7 contains a variable fragment (Fv fragment) 9 formed of the V_{L} and V_{H}. The Fv fragment 9 accommodates the antigen-binding site, namely paratope. The paratope can be at the tip of the arm of the Y-shaped rabbit mAb 1.

Each of the variable regions, V_{H} and V_{L}, can include complementarity-determining regions (CDRs) and framework regions (FRs). The CDRs determine the specificity and binding affinity of the Y-shaped rabbit mAb 1. The CDRs contain the antigen-contacting residues and are responsible for the ability of the rabbit mAb 1 to recognize and contact the S1. The Y-shaped rabbit mAb 1 can include six (6) CDRs, three of which are in the V_{H}, namely V_{H} CDR1, V_{H} CDR2, and V_{H} CDR3, and the other three of which are in the V_{L}, namely V_{L} CDR1, V_{L} CDR3, and V_{L} CDR3.

The CDRs of V_{H} and V_{L} can each be separated by the FRs. The FRs are conserved regions in sequence structures. The FRs can generally act as a scaffold so that the CDRs can adopt three-dimensional structures capable of directly contacting the antigen, i.e., the S1. The three-dimensional structure of the FRs can be conserved across different antibodies. In an embodiment, the CDRs of the Y-shaped rabbit mAb 1 can be grafted into FRs of another antibody from other species while retaining their ability to bind the spike S1 protein, forming a mosaic antibody. In an embodiment, the CDRs of the Y-shaped rabbit mAb 1 are grafted into FRs of a human antibody, forming a humanized antibody against the S1.

The Fc fragment 8 can be formed of C_{H}2 and C_{H}3 from the two heavy chains 2. In an embodiment, the Fc fragment 8 can include three constant domains. As the Fc fragment 8 can be composed of the constant domains of the heavy chains, the classes of the heavy chains can be used to categorize the antibody. The Fc fragment 8 of the Y-shaped rabbit mAb 1 generally does not involve binding the antigen. In an embodiment, the Fc fragment 8 can play a role in modulating immune cell activity, for example, by binding to a specific class of Fc receptors or other immune molecules such as complement proteins. In an embodiment, the Fc fragment 8 can play a role in generating an appropriate immune response when the CDRs bind to the antigen. In an embodiment, the Fc fragment 8 can mediate different physiological effects that can include but not limited to recognition of opsonized particles when binding to FcγR, degranulation of mast cells, basophils, and eosinophils when binding to Fcε receptors, lysis of cells or complement-dependent cytotoxicity when binding to complement proteins, antibody-dependent cellular cytotoxicity (ADCC), antibody-dependent cell phagocytosis (ADCP), interaction with the neonatal Fc receptor (FcRn) to slow down antibody degradation and extend its serum half-life.

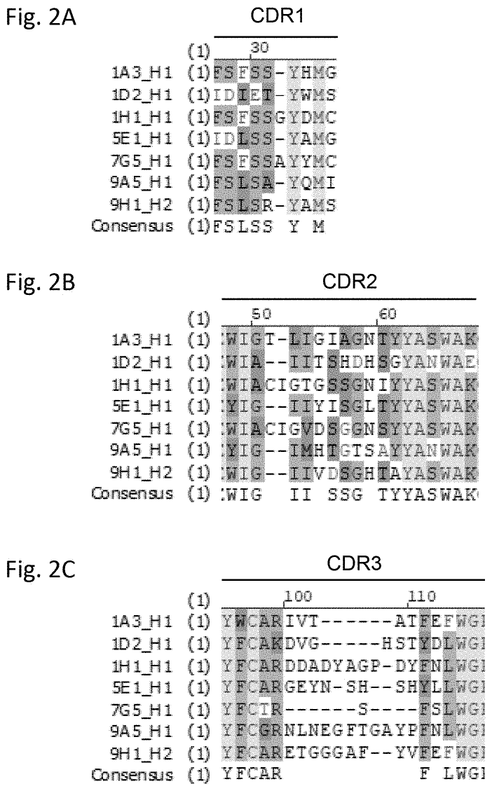

FIGS. 2A, 2B, and 2C respectively show sequence alignments of CDR1, CDR2, and CDR3 of the heavy chain variable regions (V_{H}) of the rabbit mAb against the spike S1 of SARS-CoV-2, in accordance with embodiments, including 1A3, 1D2, 1H1, 5E1, 7G5, 9A5, and 9H1.

Referring to FIG. 2A, the V_{H} CDR1s of 1A3, 1D2, 1H1, 5E1, 7G5, 9A5, and 9H1 can have a length of about 9-10 amino acids. The amino acid sequence of the V_{H} CDR1 of 1A3 can include or consist of FSFSSYHMG (SEQ ID NO: 1). The amino acid sequence of the V_{H} CDR1 of 1D2 can include or consist of IDIETYWMS (SEQ ID NO: 2). The amino acid sequence of the V_{H} CDR1 of 1H1 can include or consist of FSFSSGYDMC (SEQ ID NO: 3). The amino acid sequence of the V_{H} CDR1 of 5E1 can include or consist of IDLSSYAMG (SEQ ID NO: 4). The amino acid sequence of the V_{H} CDR1 of 7G5 can include or consist of FSFSSAYYMC (SEQ ID NO: 5). The amino acid sequence of V_{H} CDR1 of 9A5 can include or consist of FSLSAYQMI (SEQ ID NO: 6). The amino acid sequence of V_{H} CDR1 of 9H1 can include or consist of FSLSRYAMS (SEQ ID NO: 7). The V_{H} CDR1s of 1A3, 1D2, 1H1, 5E1, 7G5, 9A5, and 9H1 have a consensus sequence formula of FSLSS[ ]Y[ ]MC. The pair of square brackets “[ ]” represents a single position in a protein sequence.

Referring to FIG. 2B, the V_{H} CDR2s of 1A3, 1D2, 1H1, 5E1, 7G5, 9A5, and 9H1 can have a length of about 18-20 amino acids. The amino acid sequence of the V_{H} CDR2 of 1A3 can include or consist of WIGTLIGIAGNTYYASWAK (SEQ ID NO: 8). The amino acid sequence of the V_{H} CDR2 of 1D2 can include or consist of WIAIITSHDHSGYANWAE (SEQ ID NO: 9). The amino acid sequence of the V_{H} CDR2 of 1H1 can include or consist of WIACIGTGSSGNIYYASWAK (SEQ ID NO: 10). The amino acid sequence of the V_{H} CDR2 of 5E1 can include or consist of YIGIIYISGLTYYASWAK (SEQ ID NO: 11). The amino acid sequence of the V_{H} CDR2 of 7G5 can include or consist of WIACIGVDSGGNSYYASWAK (SEQ ID NO: 12). The amino acid sequence of V_{H} CDR2 of 9A5 can include or consist of YIGIMHTGTSAYYANWAK (SEQ ID NO: 13). The amino acid sequence of V_{H} CDR2 of 9H1 can include or consist of WIGIIVDSGHTAYASWAK (SEQ ID NO: 14). The V_{H} CDR2s of 1A3, 1D2, 1H1, 5E1, 7G5, 9A5, and 9H1 have a consensus sequence formula of WIG[ ][ ]II[ ]SSG[ ]TYYASWAK.

Referring to FIG. 2C, the V_{H} CDR3s of 1A3, 1D2, 1H1, 5E1, 7G5, 9A5, and 9H1 can have a length of about 12-22 amino acids. The amino acid sequence of the V_{H} CDR3 of 1A3 can include or consist of YWCARIVTATFEFWG (SEQ ID NO: 15). The amino acid sequence of the V_{H} CDR3 of 1D2 can include or consist of YFCAKDVGHSTYDLWG (SEQ ID NO: 16). The amino acid sequence of the V_{H} CDR3 of 1H1 can include or consist of YFCARDDADYAGPDYFNLWG (SEQ ID NO: 17). The amino acid sequence of the V_{H} CDR3 of 5E1 can include or consist of YFCARGEYNSHSHYLLWG (SEQ ID NO: 18). The amino acid sequence of the V_{H} CDR3 of 7G5 can include or consist of YFCTRSFSLWG (SEQ ID NO: 19). The amino acid sequence of V_{H} CDR3 of 9A5 can include or consist of YFCGRNLNEGFTGAYPFNLWG (SEQ ID NO: 20). The amino acid sequence of V_{H} CDR3 of 9H1 can include or consist of YFCARETGGGAFYVFEFWG (SEQ ID NO: 21). The V_{H} CDR3s of 1A3, 1D2, 1H1, 5E1, 7G5, 9A5, and 9H1 have a consensus sequence formula of YFCAR[ ][ ][ ][ ][ ][ ][ ][ ][ ][ ][ ]F[ ]LWGI.

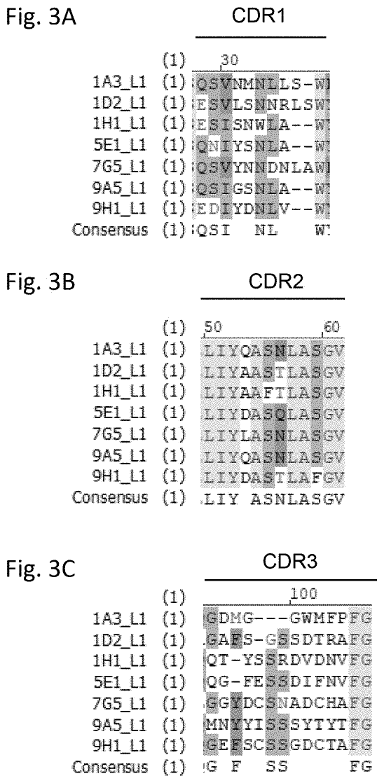

FIGS. 3A, 3B, and 3C respectively show sequence alignments of CDR1, CDR2, and CDR3 of the light chain variable regions (V_{L}) of the rabbit mAb against the SARS-CoV-2 S1, in accordance with embodiments, including 1A3, 1D2, 1H1, 5E1, 7G5, 9A5, and 9H1.

Referring to FIG. 3A, the V_{L}CDR1s of 1A3, 1D2, 1H1, 5E1, 7G5, 9A5, and 9H1 can have a length of about 9-11 amino acids. The amino acid sequence of the V_{L} CDR1 of 1A3 can include or consist of QSVNMNLLSW (SEQ ID NO: 22). The amino acid sequence of the V_{L} CDR1 of 1D2 can include or consist of ESVLSNNRLSW (SEQ ID NO: 23). The amino acid sequence of the V_{L} CDR1 of 1H1 can include or consist of ESISNWLAW (SEQ ID NO: 24). The amino acid sequence of the V_{L} CDR1 of 5E1 can include or consist of QNIYSNLAW (SEQ ID NO: 25). The amino acid sequence of the V_{L} CDR1 of 7G5 can include or consist of QSVYNNDNLAW (SEQ ID NO: 26). The amino acid sequence of V_{L} CDR1 of 9A5 can include or consist of WSIGSNLAW (SEQ ID NO: 27). The amino acid sequence of V_{L} CDR1 of 9H1 can include or consist of EDIYDNLVW (SEQ ID NO: 28). The V_{L} CDR1s of 1A3, 1D2, 1H1, 5E1, 7G5, 9A5, and 9H1 have a sequence consensus formula of QSI[ ][ ]NL[ ][ ][ ]W. The pair of square brackets “[ ]” represents a single position in a protein sequence.

Referring to FIG. 3B, the V_{L} CDR2s of 1A3, 1D2, 1H1, 5E1, 7G5, 9A5, and 9H1 can have a length of about 12 amino acids. The amino acid sequence of the V_{L} CDR2 of 1A3 can include or consist of LIYQASNLASGV (SEQ ID NO: 29). The amino acid sequence of the V_{L} CDR2 of 1D2 can include or consist of LIYAASTLASGV (SEQ ID NO: 30). The amino acid sequence of the V_{L} CDR2 of 1H1 can include or consist of LIYAAFTLASGV (SEQ ID NO: 31). The amino acid sequence of the V_{L} CDR2 of 5E1 can include or consist of LIYDASQLASGV (SEQ ID NO: 32). The amino acid sequence of the V_{L} CDR2 of 7G5 can include or consist of LIYLASNLASGV (SEQ ID NO: 33). The amino acid sequence of V_{L} CDR2 of 9A5 can include or consist of LIYQASNLASGV (SEQ ID NO: 34). The amino acid sequence of V_{L} CDR2 of 9H1 can include or consist of LIYDASTLAFGV (SEQ ID NO: 35). The V_{L} CDR2s of 1A3, 1D2, 1H1, 5E1, 7G5, 9A5, and 9H1 have a consensus sequence formula of LIY[ ]ASNLASGV.

Referring to FIG. 3C, the V_{L} CDR3s of 1A3, 1D2, 1H1, 5E1, 7G5, 9A5, and 9H1 can have a length of about 11-14 amino acids. The amino acid sequence of the V_{L} CDR3 of 1A3 can include or consist of GDMGGWMFPFG (SEQ ID NO: 36). The amino acid sequence of the V_{L} CDR3 of 1D2 can include or consist of GAFSGSSDTRAFG (SEQ ID NO: 37). The amino acid sequence of the V_{L} CDR3 of 1H1 can include or consist of QTYSSRDVDNVFG (SEQ ID NO: 38). The amino acid sequence of the V_{L} CDR3 of 5E1 can include or consist of QGFESSDIFNVFG (SEQ ID NO: 39). The amino acid sequence of the V_{L} CDR3 of 7G5 can include or consist of GGYDCSNADCHAFG (SEQ ID NO: 40). The amino acid sequence of V_{L} CDR3 of 9A5 can include or consist of MNYYISSSYTYTFG (SEQ ID NO: 41). The amino acid sequence of V_{L} CDR3 of 9H1 can include or consist of GEFSCSSGDCTAFG (SEQ ID NO: 42). The V_{L} CDR3s of 1A3, 1D2, 1H1, 5E1, 7G5, 9A5, and 9H1 have a consensus sequence formula of G[ ]F[ ][ ]SS[ ][ ][ ][ ][ ]FG.

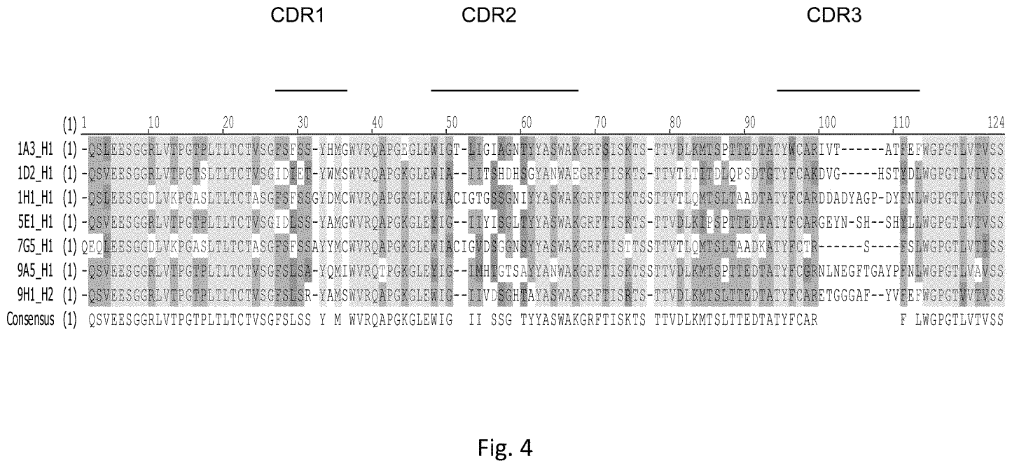

Referring to FIG. 4, FIG. 4 shows a sequence alignment of variable regions of heavy chains of the rabbit mAb against the spike S1 of SARS-CoV-2, in accordance with embodiments, including 1A3, 1D2, 1H1, 5E1, 7G5, 9A5, and 9H1.

The amino acid sequence of the V_{H} of 1A3 can include or consist of

(SEQ ID NO: 43)
QSLEESGGRLVTPGTPLTLTCTVSGFSFSSYHMGW
VRQAPGEGLEWIGTLIGIAGNTYYASWAKGRFSIS
KTSTTVDLKMTSPTTEDTATYWCARIVTATFEFWG
PGTLVTVSS.

The amino acid sequence of the V_{H} of 1D2 can include or consist of

(SEQ ID NO: 44)
QSVEESGGRLVTPGTSLTLTCTVSGIDIETYWMSW
VRQAPGKGLEWIAIITSHDHSGYANWAEGRETISK
TSTTVTLTITDLQPSDTGTYFCAKDVGHSTYDLWG
PGTLVTVSS.

The amino acid sequence of the V_{H} of 1h1 can include or consist of

(SEQ ID NO: 45)
QSLEESGGDLVKPGASLTLTCTASGFSFSSGYDMCW
VRQAPGKGLEWIACIGTGSSGNIYYASWAKGRFTI
SKTSSTTVTLQMTSLTAADTATYFCARDDADYAGP
DYFNLWGPGTLVTVSS.

The amino acid sequence of the V_{H} of 5E1 can include or consist of

(SEQ ID NO: 46)
QSVEESGGRLVTPGTPLTLTCTVSGIDLSSYAMGW
VRQAPGKGLEYIGIIYISGLTYYASWAKGRFTISK
TSTTVDLKIPSPTTEDTATYFCARGEYNSHSHYLL
WGPGTLVTVSS.

The amino acid sequence of the V_{H} of 7G5 can include or consist of

(SEQ ID NO: 47)
QEQLEESGGDLVKPGASLTLTCTASGFSFSSAYYM
CWVRQAPGKGLEWIACIGVDSGGNSYYASWAKGRF
TISTTSSTTVTLQMTSLTAADKATYFCTRSFSLWG
PGTLVTISS.

The amino acid sequence of the V_{H} of 9A5 can include or consist of

(SEQ ID NO: 48)
QSVEESGGRLVTPGTPLTLTCTVSGFSLSAYQMIW
VRQTPGKGLEYIGIMHTGTSAYYANWAKGRFTISK
TSSTTVDLKMTSPTTEDTATYFCGRNLNEGFTGAY
PFNLWGPGTLVAVSS.

The amino acid sequence of the V_{H} of 9H1 can include or consist of

(SEQ ID NO: 49)
QSVEESGGRLVTPGTPLTLTCTVSGFSLSRYAMSW
VRQAPGKGLEWIGIIVDSGHTAYASWAKGRFTISR
TSTTVDLKMTSLTTEDTATYFCARETGGGAFYVFE
FWGPGTVVTVSS.

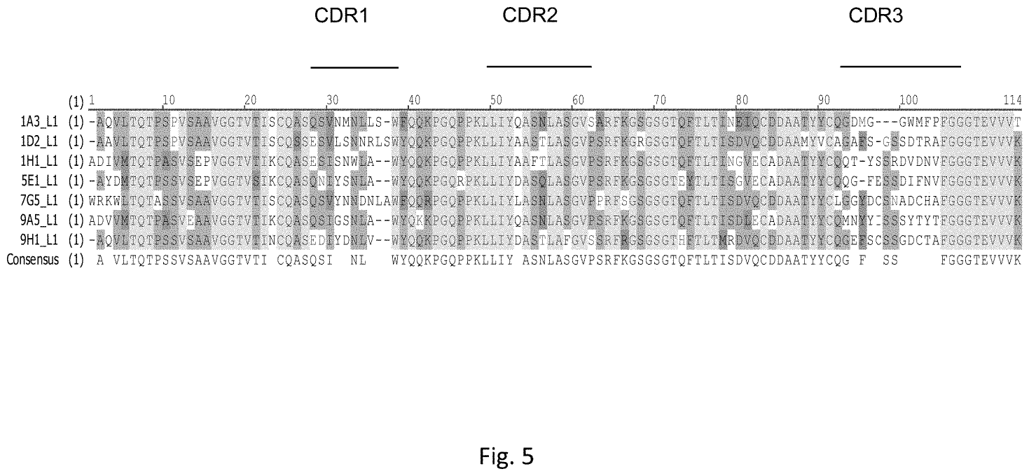

Referring to FIG. 5, FIG. 5 shows a sequence alignment of variable regions of light chains of the rabbit mAb against the spike S1 of SARS-CoV-2, in accordance with embodiments including 1A3, 1D2, 1H1, 5E1, 7G5, 9A5, and 9H1.

The amino acid sequence of the V_{L} of 1A3 can include or consist of

(SEQ ID NO: 50)
AQVLTQTPSPVSAAVGGTVTISCQASQSVNMNLLS
WFQQKPGQPPKLLIYQASNLASGVSARFKGSGSGT
QFTLTINEIQCDDAATYYCQGDMGGWMFPFGGGTE
VVVT.

The amino acid sequence of the V_{L} of 1D2 can include or consist of

(SEQ ID NO: 51)
AAVLTQTPSPVSAAVGGTVTISCQSSESVLSNNRL
SWYQQKPGQPPKLLIYAASTLASGVPSRFKGRGSG
TQFTLTISDVQCDDAAMYVCAGAFSGSSDTRAFGG
GTEVVVK.

The amino acid sequence of the V_{L} of 1H1 can include or consist of

(SEQ ID NO: 52)
ADIVMTQTPASVSEPVGGTVTIKCQASESISNWLA
WYQQKPGQPPKLLIYAAFTLASGVPSRFKGSGSGT
QFTLTINGVECADAATYYCQQTYSSRDVDNVFGGG
TEVVVK.

The amino acid sequence of the V_{L} of 5E1 can include or consist of

(SEQ ID NO: 53)
AYDMTQTPSSVSEPVGGTVSIKCQASQNIYSNLAW
YQQKPGQRPKLLIYDASQLASGVPSRFKGSGSGTE
YTLTISGVECADAATYYCQQGFESSDIFNVFGGGT
EVVVK.

The amino acid sequence of the V_{L} of 7G5 can include or consist of

(SEQ ID NO: 54)
WRKWLTQTASSVSAAVGGTVTISCQASQSVYNNDN
LAWFQQRPGQPPKLLIYLASNLASGVPPRFSGSGS
GTQFTLTISDVQCDDAATYYCLGGYDCSNADCHAF
GGGTEVVVK.

The amino acid sequence of the V_{L} of 9A5 can include or consist of

(SEQ ID NO: 55)
ADVVMTQTPASVEAAVGGTVTIKCQASQSIGSNLA
WYQKKPGQPPKLLIYQASNLASGVPSRFKGSGSGT
QFTLTISDLECADAATYYCQMNYYISSSYTYTFGG
GTEVVVK.

The amino acid sequence of the V_{L} of 9H1 can include or consist of

(SEQ ID NO: 56)
AQVLTQTPSSVSAAVGGTVTINCQASEDIYDNLVWYQQKPGQPPKLLIYDA
STLAFGVSSRFRGSGSGTHFTLTMRDVQCDDAATYYCQGEFSCSSGDCTAF
GGGTEVVVK.

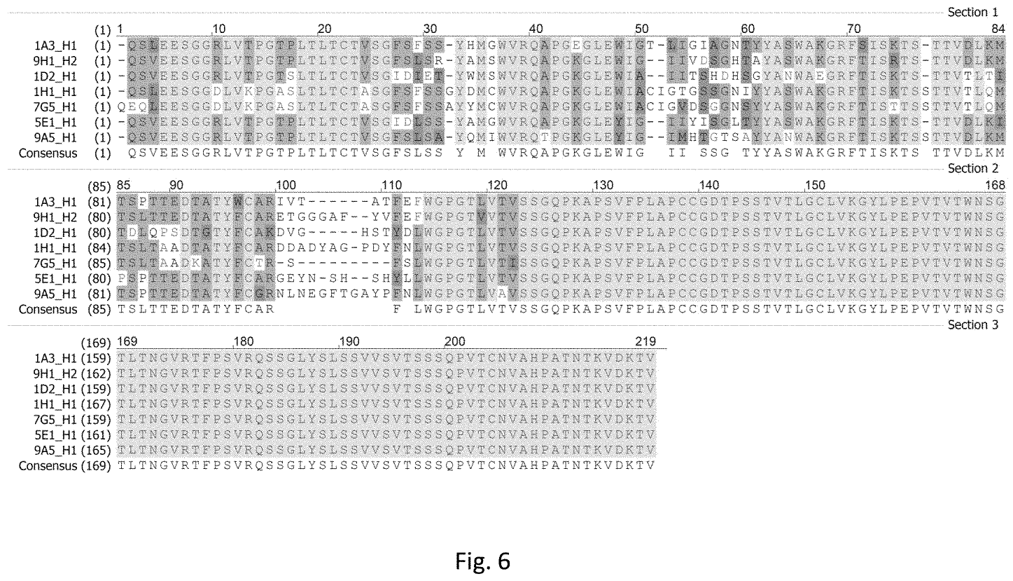

Referring to FIG. 6, FIG. 6 shows a sequence alignment of heavy chains of Fab fragments of the rabbit mAb against the spike S1 of SARS-CoV-2, in accordance with embodiments including 1A3, 1D2, 1H1, 5E1, 7G5, 9A5, and 9H1.

The amino acid sequence of the heavy chain of the Fab fragment of 1A3 can include or consist of

(SEQ ID NO: 57)
QSLEESGGRLVTPGTPLTLTCTVSGFSFSSYHMGWVRQAPGEGLEWIGTLI
GIAGNTYYASWAKGRFSISKTSTTVDLKMTSPTTEDTATYWCARIVTATFE
FWGPGTLVTVSSGQPKAPSVFPLAPCCGDTPSSTVTLGCLVKGYLPEPVTV
TWNSGTLTNGVRTFPSVRQSSGLYSLSSVVSVTSSSQPVTCNVAHPATNTK
VDKTV.

The amino acid sequence of the heavy chain of the Fab fragment of 1D2 can include or consist of

(SEQ ID NO: 58)
QSVEESGGRLVTPGTSLTLTCTVSGIDIETYWMSWVRQAPGKGLEWIAIIT
SHDHSGYANWAEGRFTISKTSTTVTLTITDLQPSDTGTYFCAKDVGHSTYD
LWGPGTLVTVSSGQPKAPSVFPLAPCCGDTPSSTVTLGCLVKGYLPEPVTV
TWNSGTLTNGVRTFPSVRQSSGLYSLSSVVSVTSSSQPVTCNVAHPATNTK
VDKTV.

The amino acid sequence of the heavy chain of the Fab fragment of 1H1 can include or consist of

(SEQ ID NO: 59)
QSLEESGGDLVKPGASLTLTCTASGFSFSSGYDMCWVRQAPGKGLEWIACI
GTGSSGNIYYASWAKGRFTISKTSSTTVTLQMTSLTAADTATYFCARDDAD
YAGPDYFNLWGPGTLVTVSSGQPKAPSVFPLAPCCGDTPSSTVTLGCLVKG
YLPEPVTVTWNSGTLTNGVRTFPSVRQSSGLYSLSSVVSVTSSSQPVTCNV
AHPATNTKVDKTV.

The amino acid sequence of the heavy chain of the Fab fragment of 5E1 can include or consist of

(SEQ ID NO: 60)
QSVEESGGRLVTPGTPLTLTCTVSGIDLSSYAMGWVRQAPGKGLEYIGIIY
ISGLTYYASWAKGRFTISKTSTTVDLKIPSPTTEDTATYFCARGEYNSHSH
YLLWGPGTLVTVSSGQPKAPSVFPLAPCCGDTPSSTVTLGCLVKGYLPEPV
TVTWNSGTLTNGVRTFPSVRQSSGLYSLSSVVSVTSSSQPVTCNVAHPATN
TKVDKTV.

The amino acid sequence of the heavy chain of the Fab fragment of 7G5 can include or consist of

(SEQ ID NO: 61)
QEQLEESGGDLVKPGASLTLTCTASGFSFSSAYYMCWVRQAPGKGLEWIAC
IGVDSGGNSYYASWAKGRFTISTTSSTTVTLQMTSLTAADKATYFCTRSFS
LWGPGTLVTISSGQPKAPSVFPLAPCCGDTPSSTVTLGCLVKGYLPEPVTV
TWNSGTLTNGVRTFPSVRQSSGLYSLSSVVSVTSSSQPVTCNVAHPATNTK
VDKTV.

The amino acid sequence of the heavy chain of the Fab fragment of 9A5 can include or consist of

(SEQ ID NO: 62)
QSVEESGGRLVTPGTPLTLTCTVSGFSLSAYQMIWVRQTPGKGLEYIGIMH
TGTSAYYANWAKGRFTISKTSSTTVDLKMTSPTTEDTATYFCGRNLNEGFT
GAYPFNLWGPGTLVAVSSGQPKAPSVFPLAPCCGDTPSSTVTLGCLVKGYL
PEPVTVTWNSGTLTNGVRTFPSVRQSSGLYSLSSVVSVTSSSQPVTCNVAH
PATNTKVDKTV.

The amino acid sequence of the heavy chain of the Fab fragment of 9H1 can include or consist of

(SEQ ID NO: 63)
QSVEESGGRLVTPGTPLTLTCTVSGFSLSRYAMSWVRQAPGKGLEWIGIIV
DSGHTAYASWAKGRFTISRTSTTVDLKMTSLTTEDTATYFCARETGGGAFY
VFEFWGPGTVVTVSSGQPKAPSVFPLAPCCGDTPSSTVTLGCLVKGYLPEP
VTVTWNSGTLTNGVRTFPSVRQSSGLYSLSSVVSVTSSSQPVTCNVAHPAT
NTKVDKTV.

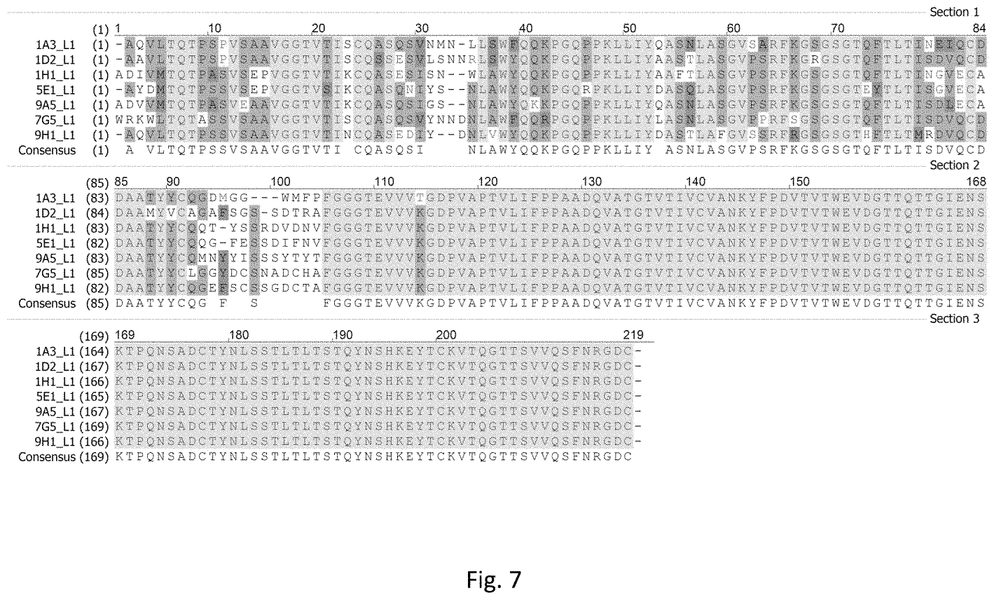

Referring to FIG. 7, FIG. 7 shows a sequence alignment of light chains of Fab fragments of the rabbit mAb against the spike S1 of SARS-CoV-2, in accordance with embodiments including 1A3, 1D2, 1H1, 5E1, 7G5, 9A5, and 9H1.

The amino acid sequence of the light chain of the Fab fragment of 1A3 can include or consist of

(SEQ ID NO: 64)
AQVLTQTPSPVSAAVGGTVTISCQASQSVNMNLLSWFQQKPGQPPKLLIYQ
ASNLASGVSARFKGSGSGTQFTLTINEIQCDDAATYYCQGDMGGWMFPFGG
GTEVVVTGDPVAPTVLIFPPAADQVATGTVTIVCVANKYFPDVTVTWEVDG
TTQTTGIENSKTPQNSADCTYNLSSTLTLTSTQYNSHKEYTCKVTQGTTSV
VQSFNRGDC.

The amino acid sequence of the light chain of the Fab fragment of 1D2 can include or consist of

(SEQ ID NO: 65)
AAVLTQTPSPVSAAVGGTVTISCQSSESVLSNNRLSWYQQKPGQPPKLLIY
AASTLASGVPSRFKGRGSGTQFTLTISDVQCDDAAMYVCAGAFSGSSDTRA
FGGGTEVVVKGDPVAPTVLIFPPAADQVATGTVTIVCVANKYFPDVTVTWE
VDGTTQTTGIENSKTPQNSADCTYNLSSTLTLTSTQYNSHKEYTCKVTQGT
TSVVQSFNRGDC.

The amino acid sequence of the light chain of the Fab fragment of 1H1 can include or consist of

(SEQ ID NO: 66)
ADIVMTQTPASVSEPVGGTVTIKCQASESISNWLAWYQQKPGQPPKLLIYA
AFTLASGVPSRFKGSGSGTQFTLTINGVECADAATYYCQQTYSSRDVDNVF
GGGTEVVVKGDPVAPTVLIFPPAADQVATGTVTIVCVANKYFPDVTVTWEV
DGTTQTTGIENSKTPQNSADCTYNLSSTLTLTSTQYNSHKEYTCKVTQGTT
SVVQSFNRGDC.

The amino acid sequence of the light chain of the Fab fragment of 5E1 can include or consist of

(SEQ ID NO: 67)
AYDMTQTPSSVSEPVGGTVSIKCQASQNIYSNLAWYQQKPGQRPKLLIYDA
SQLASGVPSRFKGSGSGTEYTLTISGVECADAATYYCQQGFESSDIFNVFG
GGTEVVVKGDPVAPTVLIFPPAADQVATGTVTIVCVANKYFPDVTVTWEVD
GTTQTTGIENSKTPQNSADCTYNLSSTLTLTSTQYNSHKEYTCKVTQGTTS
VVQSFNRGDC.

The amino acid sequence of the light chain of the Fab fragment of 7G5 can include or consist of

(SEQ ID NO: 68)
WRKWLTQTASSVSAAVGGTVTISCQASQSVYNNDNLAWFQQRPGQPPKLLI
YLASNLASGVPPRFSGSGSGTQFTLTISDVQCDDAATYYCLGGYDCSNADC
HAFGGGTEVVVKGDPVAPTVLIFPPAADQVATGTVTIVCVANKYFPDVTVT
WEVDGTTQTTGIENSKTPQNSADCTYNLSSTLTLTSTQYNSHKEYTCKVTQ
GTTSVVQSFNRGDC.

The amino acid sequence of the light chain of the Fab fragment of 9A5 can include or consist of

(SEQ ID NO: 69)
ADVVMTQTPASVEAAVGGTVTIKCQASQSIGSNLAWYQKKPGQPPKLLIYQ
ASNLASGVPSRFKGSGSGTQFTLTISDLECADAATYYCQMNYYISSSYTYT
FGGGTEVVVKGDPVAPTVLIFPPAADQVATGTVTIVCVANKYFPDVTVTWE
VDGTTQTTGIENSKTPQNSADCTYNLSSTLTLTSTQYNSHKEYTCKVTQGT
TSVVQSFNRGDC.

The amino acid sequence of the light chain of the Fab fragment of 9H1 can include or consist of

(SEQ ID NO: 70)
AQVLTQTPSSVSAAVGGTVTINCQASEDIYDNLVWYQQKPGQPPKLLIYDA
STLAFGVSSRFRGSGSGTHFTLTMRDVQCDDAATYYCQGEFSCSSGDCTAF
GGGTEVVVKGDPVAPTVLIFPPAADQVATGTVTIVCVANKYFPDVTVTWEV
DGTTQTTGIENSKTPQNSADCTYNLSSTLTLTSTQYNSHKEYTCKVTQGTT
SVVQSFNRGDC.

The rabbit mAb against the SARS-CoV-2 S1 can also be an antigen-binding portion of the Y-shaped antibodies disclosed herein. In an embodiment, the rabbit mAb against the SARS-CoV-2 S1 can be the Fab fragment 7—a monovalent fragment formed of the V_{L}, V_{H}, C_{L}, and C_{H1} domains. In an embodiment, the rabbit mAb against the SARS-CoV-2 S1 can be an F(ab′)₂ fragment that is a bivalent fragment including the two Fab fragments 7 linked by, e.g., a disulfide bridge of the hinge region 10. In an embodiment, the rabbit mAb against the SARS-CoV-2 S1 can be an Fd fragment formed of the V_{H} and C_{H1} domains. In an embodiment, the rabbit mAb against the SARS-CoV-2 S1 can be the Fv fragment 9 formed of the V_{L} and V_{H} domains. In an embodiment, the rabbit mAb against the SARS-CoV-2 S1 can be an isolated complementarity-determining region.

The rabbit mAb against the SARS-CoV-2 S1 can also encompass structures derived from the embodiments or their antigen-binding portions by genetic modification. Different genetically modified antibody structures can be generated, including but not being limited to humanized antibodies, chimeric antibodies, etc. In an embodiment, the rabbit mAb against the SARS-CoV-2 S1 can be a humanized antibody, as its protein sequence has been modified to increase its similarity to antibody variants produced naturally in humans. The protein sequence of a humanized antibody can be essentially identical to that of a human variant, despite the rabbit origin of some of its CDRs that are essential to the ability of the antibody to bind to the SARS-CoV-2 S1. In an embodiment, a humanized antibody can be created by inserting CDRs of a non-human antibody, e.g., a rabbit antibody, into a human antibody scaffold. In an embodiment, the rabbit mAb against the SARS-CoV-2 S1 can be a chimeric antibody. In an embodiment, the chimeric antibody can be an antibody made by transplanting variable regions of the heavy and light chains from the Y-shaped antibodies herein onto constant regions from another species such as a human. In an embodiment, the chimeric antibody is an antibody made by fusing Fab of one of the rabbit mAbs against the SARS-CoV-2 S1 disclosed herein with Fc of a human antibody. In an embodiment, the rabbit mAb against the SARS-CoV-2 S1 can be a single-chain Fv (scFv). Although the two domains of the Fv fragment, namely V_{L} and V_{H}, are coded by separate genes, they can be joined, using recombinant methods, by a synthetic linker to form the scFv. In an embodiment, the genetic modification can be performed in accordance with methods known in the art, and the genetically modified antibody structures can be screened in the same manner as the full-length antibodies.

The antibody disclosed herein can also encompass structures derived from the embodiments disclosed herein and their antigen-binding portions by chemical modification. In an embodiment, the chemical modification can be a chemical crosslinking. In an embodiment, one or more conjugates can be covalently linked to or non-covalently attached to the antibody. In an embodiment, the conjugate can be a molecular label covalently attached to the antibody to facilitate the detection of its antigens. The conjugates can be any suitable small molecules. The small molecule can include but is not limited to, for example, biotin, streptavidin, and/or fluorescent dye. The fluorescent dye can be any suitable fluorescent dye, including but not limited to Alexa fluors, aminomethylcoumarin (AMCA), Atto dyes, cyanine dyes, DyLight fluors, FITC, FluoProbes 647H, Rhodamine, and Texas Red. The Alexa fluors include but are not limited to Alexa Fluor 488, Alexa Fluor 555, Alexa Fluor 568, Alexa Fluor 594, Alexa Fluor 647, and Alexa Fluor 700. The Atto dyes can include but are not limited to Atto 390, Atto 488, Atto 565, Atto 633, and Atto 700. The cyanine dyes can include but are not limited to Cy3, Cy5, and Cy5.5. The DyLight dyes can include but not limited to DyLight 350, DyLight 405, DyLight 488, DyLight 550, DyLight 594, DyLight 633, DyLight 650, DyLight 680, DyLight 755, and DyLight 800. In an embodiment, the conjugates can be a tandem dye with two covalently attached fluorescence molecules. In an embodiment, one of the fluorescence molecules serves as a donor, and the other serves as an acceptor. In an embodiment, the donor and the acceptor can behave as a unique fluorophore with the donor's excitation properties and the acceptor's emission properties. The tandem dye can include but is not limited to Allophycocyanin-Cy5.5, Allophycocyanin-Cy 7, PE-Atto 594, PE-Cy 5, PE-Cy 5.5, PE-Cy 7, PE-Texas Red, PE-Alexa Fluor 647, PE-Alexa Fluor 700, PE-Alexa Fluor 750, APC-Alexa Fluor 750, and PerCP-Cy5.5.

The conjugates can also be large molecules. In an embodiment, the large molecule can be an enzyme. The enzyme can include but is not limited to alkaline phosphatase (AP), glucose oxidase (Gox), Horseradish peroxidase (HRP). In an embodiment, the large molecule can be a fluorescent protein. The fluorescent protein can include but is not limited to Allophycocyanin (APC), B-Phycoerythrin (BPE R-Phycoerythrin (R-PE), PerCP, and R-Phycocyanin (RPC). In an embodiment, the large molecule can also be an antibody whose specificity differs from that of the rabbit mAb against the SARS-CoV-2 S1, forming a tandem antibody with multiple specificities.

The rabbit mAb against the SARS-CoV-2 S1 can have various in vivo and in vitro applications, including but not limited to immunoassays, immunostaining, immunohistochemistry, diagnosis of virus infection associated with SARS-CoV-2, immuno-oncology therapies, and treatment of some infectious diseases caused by SARS-CoV-2. The immunoassays can include enzyme-linked immunosorbent assay (ELISA). The mAb against the SARS-CoV-2 S1 can be used in different forms of ELISA. In an embodiment, the rabbit mAb against the SARS-CoV-2 S1 disclosed can be used in a direct ELISA. A direct ELISA can be a plate-based immunosorbent assay intended to detect and quantify a specific antigen from or within a complex biological sample. There are varieties of methods for performing direct ELISA.

In an embodiment, the antigen, e.g., the spike S1 of SARS-CoV-2, can be immobilized or adsorbed onto a surface of a plastic plate. In an embodiment, the plastic plate can be a multi-well microtiter plate. In an embodiment, the multi-well microtiter plane can be a 96-well polystyrene plate. In an embodiment, an excessive amount of blocking protein can be added onto the surface to block all the other binding sites. In an embodiment, the blocking protein is bovine serum albumin. In an embodiment, an antibody specific for the antigen, e.g., the spike S1 of SARS-CoV-2, can be added onto the surface to form a complex with the antigen. In an embodiment, the antibody can be conjugated with an enzyme. In an embodiment, the enzyme can be HRP. After the excess conjugated antibody is washed off, the conjugated antibody bound to the antigen stays. In an embodiment, the conjugated antibody catalyzes a reaction with an added substrate, resulting in a visible colorimetric output that can be measured by a spectrophotometer or absorbance microplate reader. Direct ELISA, when compared to other forms of ELISA testing, can be performed faster because only one antibody is used, and fewer steps are required. In an embodiment, the direct ELISA can test specific antibody-to-antigen reactions and helps to eliminate cross-reactivity between other antibodies. Direct ELISA is suitable for qualitative and quantitative antigen detection in samples of interest, antibody screening, and epitope mapping.

The binding Kinetics 1A3, 1D2, 1H1, 5E1, 7G5, 9A5, and 9H1 are summarized in Table 1. As seen in Table 1, 1A3, 1D2, 1H1, 5E1, 7G5, 9A5, and 9H1 exhibit excellent binding affinity and specificity to the SARS-CoV-2 S1, as evidenced by their dissociate constants (K_{D}) of 5.37E-10, 3.55E-09, 2.06E-09, 1.38E-10, 2.14E-09, 8.70E-10, 7.69E-10, respectively.

TABLE 1
The binding Kinetics 1A3, 1D2, 1H1, 5E1, 7G5, 9A5, and 9H1

| Clones | K_{off}(1/s) | Kₒₙ(1/Ms) | K_{D}(M) |
|---|---|---|---|
| 1A3 | 9.34E−05 | 1.74E+05 | 5.37E−10 |
| 1D2 | 2.59E−04 | 7.29E+04 | 3.55E−09 |
| 1H1 | 2.73E−04 | 1.33E+05 | 2.06E−09 |
| 5E1 | 1.00E−05 | 7.23E+04 | 1.38E−10 |
| 7G5 | 2.61E−04 | 1.22E+05 | 2.14E−09 |
| 9A5 | 1.15E−04 | 1.32E+05 | 8.70E−10 |
| 9H1 | 2.50E−04 | 3.25E+05 | 7.69E−10 |

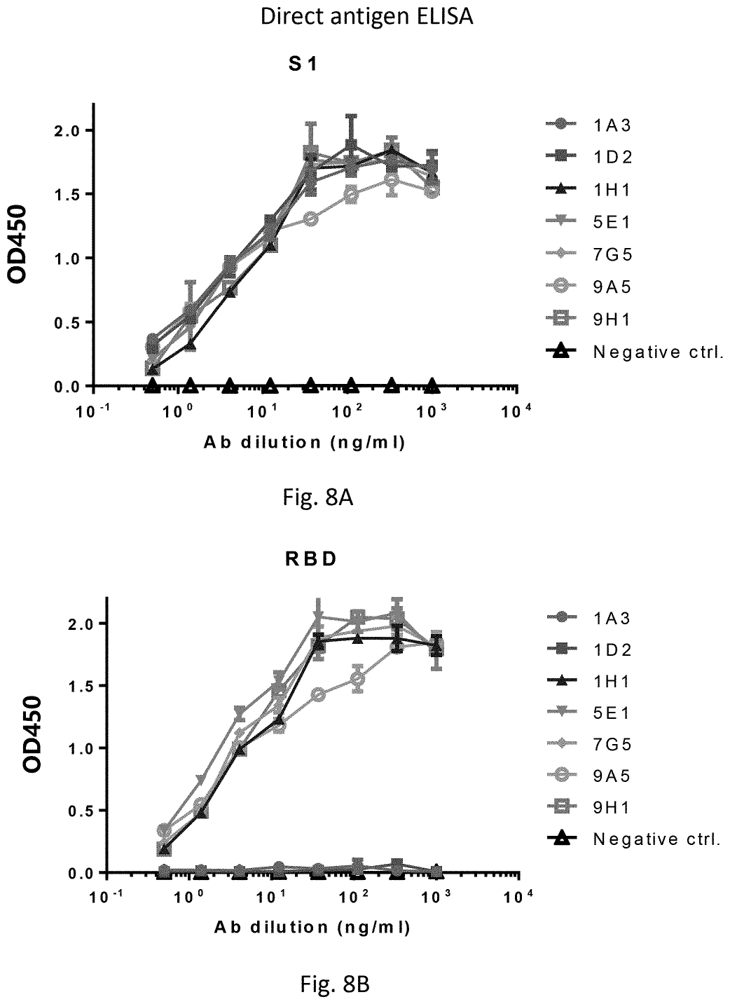

Referring to FIG. 8A, FIG. 8A shows curves of 1A3, 1D2, 1H1, 5E1, 7G5, 9A5, and 9H1 in a direct antigen ELISA for detecting the SARS-CoV-2 S1, in accordance with an embodiment. The X-axis shows antibody concentration at a unit of ng/ml, and the Y-axis shows optical density at the wavelength of 450 nm (OD₄₅₀). As seen, all the rabbit mAbs can specifically bind to the SARS-CoV-2 S1, and their binding curves exhibit excellent “S” shape in a wide range of antibody concentrations from about 1 ng/ml to about 1000 ng/ml. The negative control was conducted under the same procedures as the rabbit mAbs against the SARS-CoV-2 S1, except that a blank buffer is used instead of the rabbit mAbs against the SARS-CoV-2 S1. The blank buffer is the buffer for diluting the rabbit mAbs. Compared to the negative control that exhibits almost no OD₄₅₀, 1A3, 1D2, 1H1, 5E1, 7G5, 9A5, and 9H1 exhibit excellent detection signal, i.e., OD450, at a wide range of concentrations of the SARS-CoV-2 S1.

Referring to FIG. 8B, FIG. 8B shows curves of 1A3, 1D2, 1H1, 5E1, 7G5, 9A5, and 9H1 in a direct antigen ELISA for detecting the RBD of the SARS-CoV-2 S1, in accordance with an embodiment. The X-axis shows antibody concentration at a unit of ng/ml, and the Y-axis shows optical density at the wavelength of 450 nm (OD₄₅₀). The negative control was conducted under the same procedures as the rabbit mAbs against the SARS-CoV-2 S1, except that a blank buffer is used instead of the rabbit mAbs against the SARS-CoV-2 S1. The blank buffer is the buffer for diluting the rabbit mAbs. The negative control exhibits almost no detection signal as its OD450 is nearly 0. However, compared to the negative control, 1H1, 5E1, 7G5, 9A5, and 9H1 can produce significant OD450. 1H1, 5E1, 7G5, 9A5, and 9H1 can specifically bind to the RBD of the SARS-CoV-2 S1, as their binding curves exhibit excellent “S” shape in a wide range of antibody concentrations from about 0.5 ng/ml to about 1000 ng/ml. Contrary to 1H1, 5E1, 7G5, 9A5, and 9H1, 1A3 and 1D2 cannot specifically bind to the RBD of the SARS-CoV-2 S1 as they exhibit almost no OD450.

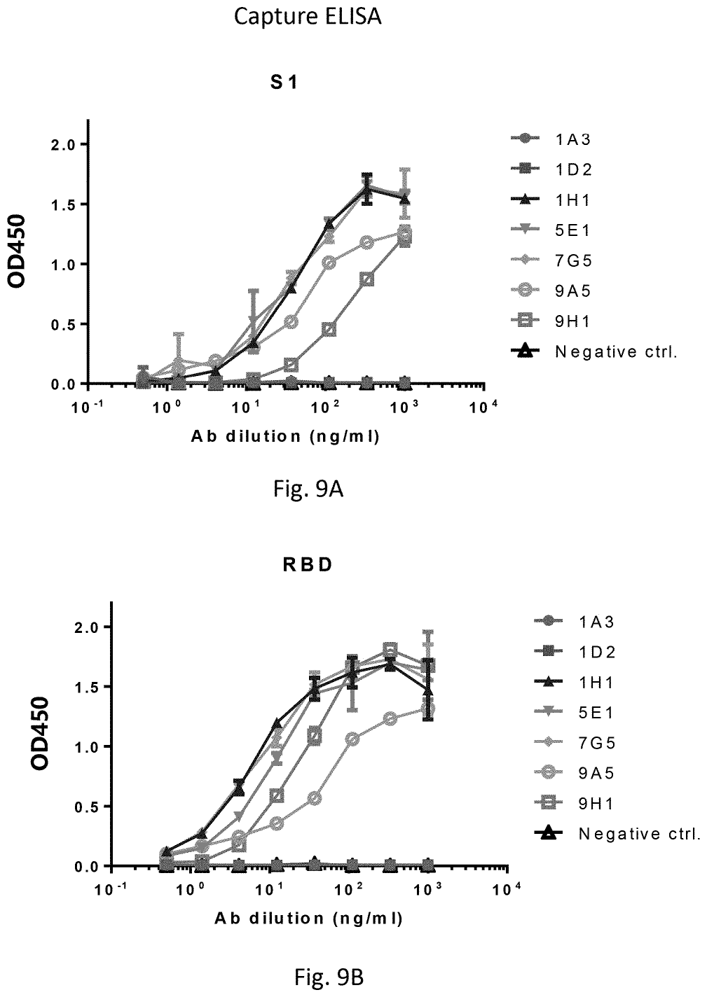

To evaluate if the antibodies can bind to the S1 in the native state, a capture ELISA is performed. The antibody is captured by an Fc coated on a plate, and then the S1 or RBD in the native state is added to the plate. The results are shown in FIG. 9A and 9B. FIG. 9A shows the capture ELISA results of 1A3, 1D2, 1H1, 5E1, 7G5, 9A5, and 9H1 for binding to the S1. As seen, 1H1, 5E1, 7G5, 9A5, and 9H1 can bind to the S1 in the native state except for 1D2 and 1A3.

Referring to FIG. 9B, FIG. 9B shows the capture ELISA results of 1A3, 1D2, 1H1, 5E1, 7G5, 9A5, and 9H1 for binding to RBD in the native state. As seen, 1H1, 5E1, 7G5, 9A5, and 9H1 can bind to the RBD in the native state except for 1D2 and 1A3. 1A3 and 1D2 can be used as detection antibody of full length S1 protein but not RBD in an ELISA assay.

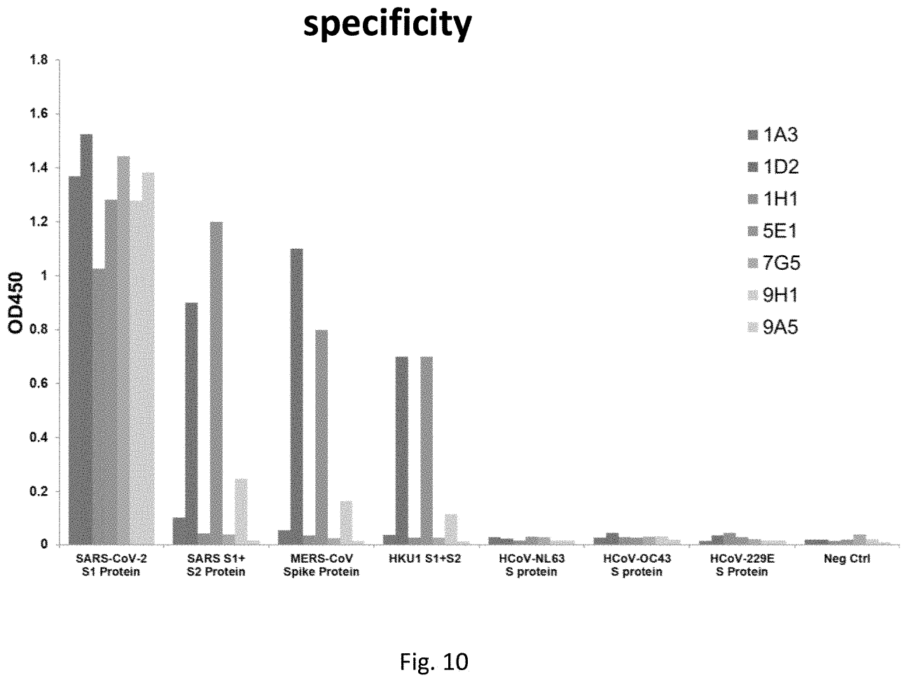

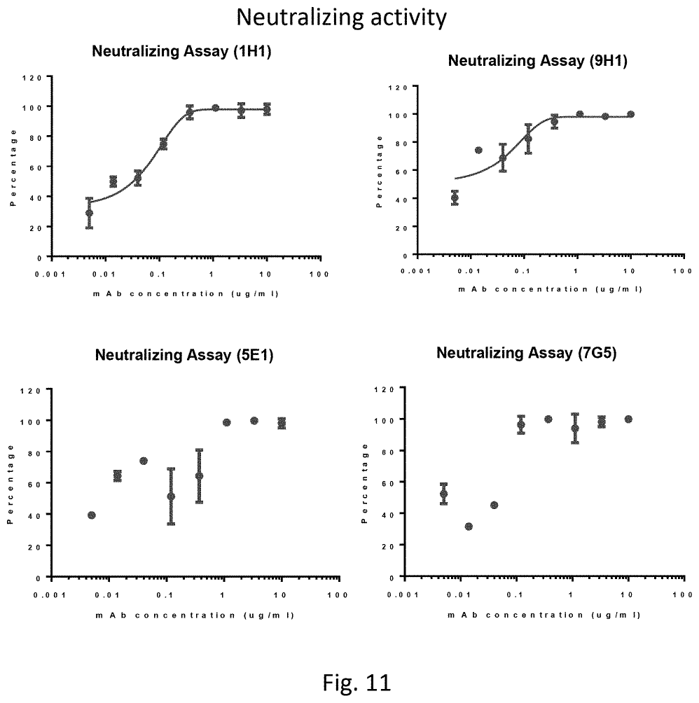

Referring to FIG. 10, FIG. 10 shows specificity of 1A3, 1D2, 1H1, 5E1, 7G5, 9H1, and 9A5 to SARS-CoV-2 (SARS-CoV-2) S1, SARS S1 and S2 protein, MERS-CoV Spike protein, HKU1 S1 and S2 protein, HcoV-NL63 S protein, HcoV-OC43 S protein, and HcoV-229E S protein, in accordance with an embodiment. The Y-axis shows optical density at 450 nm of a direct ELISA assay. Each set of columns includes seven (7) columns that are arranged in the order of 1A3, 1D2, 1H1, 5E1, 7G5, 9A5, and 9H1 along the X-axis direction to show the specificity to the SARS-CoV-2 S1, SARS S1, MERS S1, and hCov-NL63 S1, respectively. As seen, the antibodies disclosed herein exhibit the highest specificity toward the SARS-CoV-2 S1. Among the antibodies, 1A3, 1H1, 7G5, and 9A5 exhibit exclusive specificity toward the SARS-CoV-2 S1, with no significant specificity toward the SARS S1 and S2 protein, MERS-CoV Spike protein, HKU1 S1 and S2 protein, HcoV-NL63 S protein, HcoV-OC43 S protein, and HcoV-229E S protein. In contrast, 1D2, 5E1, and 9H1 exhibit specificity toward the SARS-CoV-2 S1, SARS S1 and S2 protein, MERS-CoV Spike protein, and HKU1 S1 and S2 protein in the order from highest to lowest specificity. However, 1D2, 5E1, and 9H1 exhibit no specificity toward HcoV-NL63 S protein, HcoV-OC43 S protein, and HcoV-229E S protein.

To evaluate the neutralization potency of 1H1, 9H1, 5E1, and 7G5, both pesudovirus assay and live viral neutralization assay were performed. The results of pesudovirus assay are shown in FIG. 11. As seen in FIG. 11, 1H1, 9H1, 5E1, and 7G5 can neutralize SARS-CoV-2 wild-type pseudotyped virus. Consistent with pseudovirus neutralization assay, 1H1, 9H1, 5E1, and 7G5 can neutralize live authentic SARS-CoV-2 virus, as seen in Table 2 below.

TABLE 2
Live SARS-CoV-2 virus neutralizing activity of
1H1, 9H1, 5E1, and 7G5.

| | IC50 against Sars-CoV-2 live |
|---|---|
| mAb | virus (ug/ml) |
| 9H1 | 0.026 |
| 1H1 | 0.136 |
| 5E1 | 0.512 |
| 7G5 | 0.261 |

As seen in Table 2, the half-maximal inhibitory concentration (IC50) values of 1H1, 9H1, 5E1, and 7G5 for live authentic SARS-CoV-2 virus (wild type) are 0.136 μg/ml, 0.026 μg/ml, 0.512 μg/ml, and 0.261 μg/ml, respectively.

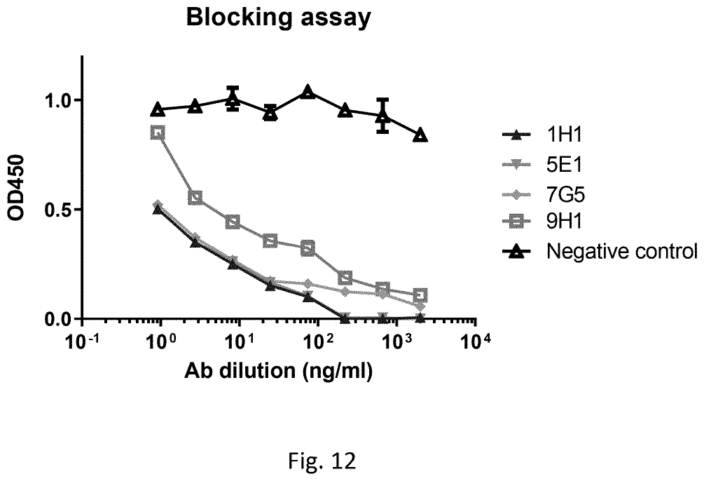

To evaluate if 1H1, 9H1, 5E1, and 7G5 can block the binding of the S1 to ACE2, a blocking assay is performed. Recombinant ACE2 was coated on an ELISA plate. Antibody was pre-incubated with the RBD domain protein at different concentrations to form antibody-RBD mixtures that were then loaded to ACE2-coated ELISA plate. The results are shown in FIG. 12. As seen, 1H1, 9H1, 5E1, and 7G5 can block the binding of the RBD domain to ACE2.

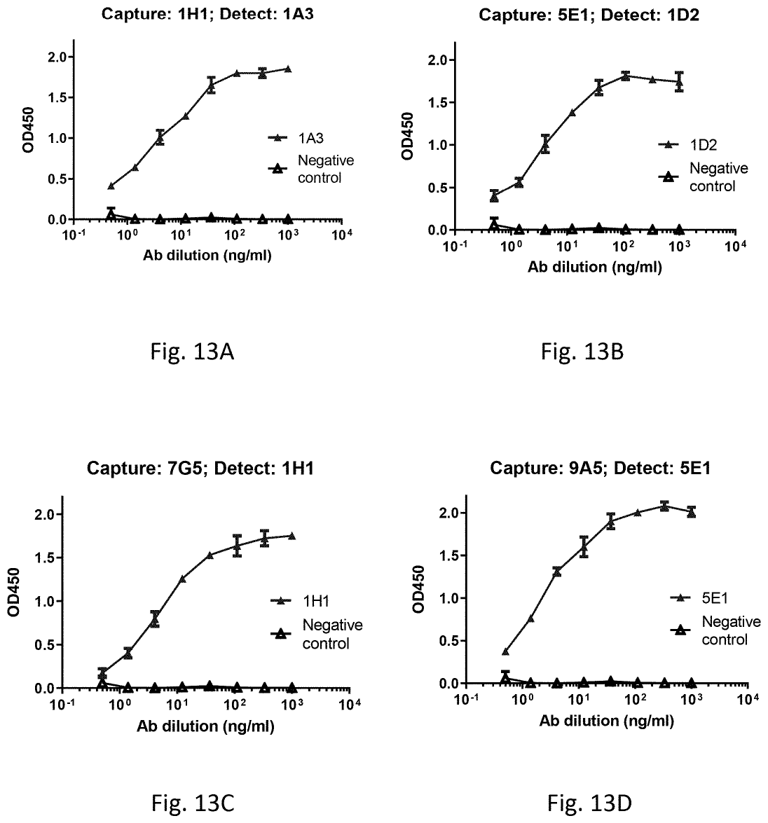

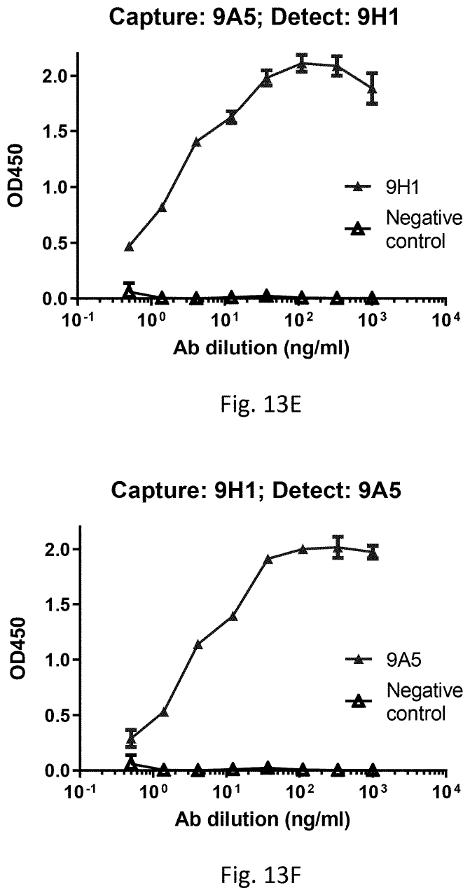

FIG. 13 A shows results of sandwich ELISA using 1H1 as the capture antibody and 1A3 as the detection antibody, in accordance with an embodiment. As seen, 1A3 can bind or detect to the S1 after the S1 is bound by 9A5.

FIG. 13 B shows results of sandwich ELISA using 5E1 as the capture antibody and 1D2 as the detection antibody, in accordance with an embodiment. As seen, 1D2 can bind or detect to the S1 after the S1 is bound by 5E1.

FIG. 13 C shows results of sandwich ELISA using 7G5 as the capture antibody and 1H1 as the detection antibody, in accordance with an embodiment. As seen, 1H1 can bind or detect the S1 after the S1 is bound by 7G5.

FIG. 13 D shows results of sandwich ELISA using 9A5 as the capture antibody and 5E1 as the detection antibody, in accordance with an embodiment. As seen, 9A5 can bind or detect the S1 after the S1 is bound by 9A5.

FIG. 13 E shows results of sandwich ELISA using 9A5 as the capture antibody and 9H1 as the detection antibody, in accordance with an embodiment. As seen, 9H1 can bind to the S1 after the S1 is bound by 9A5.

FIG. 13 F shows results of sandwich ELISA using 9H1 as the capture antibody and 9A5 as the detection antibody, in accordance with an embodiment. As seen, 9H1 can bind to the S1 after the S1 is bound by 9H1.

Referring to FIG. 11, FIG. 11 shows results of neutralizing assay of 1H1, 9H1, 5E1, and 7G5, in accordance with an embodiment. The X-axis shows antibody concentration, and the Y-axis shows the percentage of inhibition of pseudovirus infection to host cells.

### Methods

1. Generation, Isolation, and Purification of Rabbit Monoclonal Antibodies Against the SARS-CoV-2 S1.

The rabbit mAbs against the SARS-CoV-2 S1 can be generated by various techniques, including monoclonal antibody methodology, e.g., the somatic cell hybridization technique and other techniques including but not limited to viral or oncogenic transformation of B lymphocytes. In an embodiment, the recombinant rabbit mAbs are generated by single B cell-based technology.

In an embodiment, the codon-optimized RBD region, corresponding to the genomic positions 22,553 to 23,312 bp in SARS-CoV-2 (GenBank:MN908947.3), was cloned into the pcDNA3.4 expression vector. The expression construct was propagated in DH5a strain of Escherichia coli and purified using the Qiagen Plasmid Mega kit (Cat no. 10023). 36 μg purified expression construct were coated to 100 μL of 100 mg/ml gold powder (Alpha Aesar, Catalog No. 39817) that was pre-coated with 100 mg/ml spermidine (Sigma, Catalog No. S2626). DNA-coating to gold was facilitated by slowly dripping 200 μl 2.5 M CaCl₂) to the DNA mixed with gold powder, which was washed in absolute ethanol before loading to bullet tubing mounted to bullet maker (Scientz Scientific). Bullet tubing loaded with gold powder was dried by slow N₂ flow at 0.1 MPa for 10 min, and the dried bullet tubing was cut into DNA bullet.

The SARS-CoV-2 RBD DNA bullets were loaded into the bullet magazine of the SJ-500 gene gun (Scientz Scientific). The SJ-500 gene gun was fired by 4 MPa helium gas to inject DNA-coated gold powder subcutaneously at shaved abdomen skin (36 μg/immunization) of New Zealand White rabbits (4-6 weeks of age). The DNA immunization was performed three times on each rabbit at days 0, 7, and 21. Then, the rabbits were boosted with 100 μg SARS-CoV-2 S1 protein emulsified in incomplete Freund's adjuvant (IFA) twice at day 35 and day 49 via intramuscular (i.m.) route. Two weeks later, the rabbits were boosted subcutaneously (s.c.) with 200 μg S1 protein. Pre and post-immunization sera were collected on days 0, 14, 28, 42, and 69, respectively.

The spleen was harvested from the rabbit. Fresh single splenocytes were isolated and cultured overnight in a B cell medium, e.g., a B cell medium from Yurogen Biosystems, China. A fresh single-cell suspension of solenocytes in PBS supplemented with 2% FBS and 1 mM EDTA was prepared before single-cell sorting. Splenocytes were processed using the single-B-cell based SMab® platform (Yurogen Biosystems, China) and sorted one cell per well on a FACS Aria II (BD Biosciences, USA) into 96-well plates. S1-specific primary B cells were cultured in a B cell complete medium, e.g., a rabbit B cell complete medium from Yurogen Biosystems, China, for 10-14 days at 37° C. with 5% CO₂. At the end of primary B cell culture, S1-recognizing B cell clones were identified by screening primary B cell culture supernatants against NB by direct ELISA. Positive B-cell clones were determined by OD450 nm values more than 5-fold over background noise. The variable region of IgG heavy chain and light chain from top positive clones was recovered by RT-PCR. The full-length IgG heavy and light chains of each clone were co-transfected into HEK293T cells. The supernatants containing recombinant rabbit IgG from transfected HEK293T were screened for their specificity to the S1 by ELISA.

The PCR fragments of variable regions of selected clones were cloned into pcDNA3.4 vector for antibody expression in HEK293F cells.

The rabbit monoclonal antibodies can be isolated and purified by a variety of techniques.

In an embodiment, rabbit monoclonal antibodies can be isolated from the culture supernatant from mammalian cells transfected with rabbit antibody genes, subsequently purified by Protein A affinity chromatography. The purity and function of purified rabbit monoclonal antibodies can be verified by SDS-PAGE and ELISA, respectively.

2. Preparation of Conjugated Rabbit Monoclonal Antibodies Against the SARS-CoV-2 S1.

Rabbit monoclonal antibodies were biotinylated using Pierce EZ-Link Sulfo-NHS-Biotin in accordance with the manufacturer manual. Briefly, rabbit monoclonal antibodies in 1×PBS, pH 7.4 were incubated with Sulfo-NHS-Biotin for 30 minutes at room temperature.

3. ELISA for characterization of immunized rabbit sera and monoclonal antibodies against the SARS-CoV-2 S1.

Antigens such as the SARS-CoV-2 S1 or S1 from other viruses were coated on high binding ELISA plates (e.g., Corning, Cat No: 9018) at 4° C. overnight in 1×PBS, pH7.4. Coated plates were washed three times with washing buffer (1×PBS supplemented with 0.5% (V/V) Tween-20 (Sigma, Cat. no: P9416), and blocked with blocking buffer (1×PBS supplemented with 5% (W/V) skim milk). After blocking, plates were incubated with serial diluted rabbit serum samples or monoclonal antibody for 1 hour at room temperature, followed by washing five times with the washing buffer and then incubating with goat anti-rabbit IgG antibody conjugated with HRP, e.g., HRP from Jackson Immuno Research, Cat No: 111-035-045, in the blocking buffer at 1:5000 dilutions. After washing the plates five times with the washing buffer, adding 25μ, TMB substrate (Moss INS, Cat no: TMBHK-1000) for 3 min in the dark at room temperature. Subsequently, the colorimetric reaction of TMB substrate was stopped with 20 μL 1 M H2SO4. Optical density (OD) values at 450 nm and 630 nm were measured by Epoch microplate spectrophotometer (Biotek, USA). The final value was obtained using OD450 subtracted by OD630. The serum titer was calculated as the maximum dilution where the diluted serum produces OD450 reading of 2-fold or above than that of the control sample.

4. Capture ELISA with the Rabbit mAbs Against the SARS-CoV-2 S1.

Anti-rabbit IgG Fc antibodies were coated on high binding ELISA plates at 4° C. overnight in 1×PBS, pH7.4. Coated plates were washed with Wash Buffer (1×PBS supplemented with 0.5% (V/V) Tween-20), and blocked with Blocking Buffer (1×PBS supplemented with 5% (W/V) skim milk). After blocking, plates were incubated with the rabbit Abs against the SARS-CoV-2 S1 for 1 hour at room temperature, followed by washing and then incubating with biotinylated SARS-CoV-2 S1 serial diluted at three-fold. The plates were further incubated with streptavidin conjugated to HRP. After a final wash, plates were developed using a colorimetric reaction catalyzed by HRP to see if a monoclonal antibody can or cannot capture the S1. It is appreciated that the capture ELISA can also be performed with different steps, reagents, experimental parameters from those discussed above.

5. Sandwich ELISA with the Rabbit mAbs Against the SARS-CoV-2 S1.

Capture antibodies were coated on high binding ELISA plates at 4° C. overnight in 0.02M bicarbonate buffer, pH9.4. Coated plates were washed with Wash Buffer (1×PBS supplemented with 0.5% (V/V) Tween-20) and blocked with Blocking Buffer (1×PBS supplemented with 5% (W/V) skim milk). After blocking, plates were incubated with the SARS-CoV-2 S1 serial diluted in 3 folds for 1 hour at room temperature, followed by washing and then incubating biotinylated mAbs against the SARS-CoV-2 S1. The plates were further incubated with streptavidin conjugated to HRP. After a final wash, plates were developed using a colorimetric reaction catalyzed by HRP to see if a pair of capture and detection monoclonal antibodies can or cannot bind to the SARS-CoV-2 S1 simultaneously. It is appreciated that the sandwich ELISA can also be performed with different steps, reagents, experimental parameters from those discussed above.

6. Determination of the Binding Kinetics of the Rabbit mAbs Against the SARS-CoV-2 S1.

The binding kinetics of the rabbit mAbs against the SARS-CoV-2 S1 were analyzed by surface plasmon resonance (SPR) using a BIAcore instrument with Protein A sensor chip (GE Healthcare, USA). All experiments were performed at 25° C. at a flow rate of 40 μL/min. The running buffer was degassed PBS with 0.005% Tween-20. Channel 1 was loaded with a reference antibody without specific binding to the antigens used, and channels 2, 3, and 4 were loaded with the antibody candidates. Typically, 2 μg/mL of antibody and a quick injection for 20-30 s yielded ˜150-250 response units (RU) of antibody coupling with high reproducibility. Antigen was then injected over all the channel surfaces for 5 min for an association phase followed by a 10-minute dissociation phase by buffer rinse. Multiple association/dissociation cycles were performed using antigen dilution series in the range of 1.2-100 nM, as well as a blank buffer. At the end of each cycle, regeneration was performed by a 30-second injection of glycine buffer (pH 2.0, 10 mM) and antibodies were loaded in each channel again. The kinetic curves were double reference subtracted and analyzed to calculate association rate constant, dissociation rate constant, and affinity constants using BIA evaluation 3.2 and 1:1 Langmuir model.

7. Authentic neutralization assay against live SARS-CoV-2 viruses.

The neutralization activity of mAbs against live SARS-CoV-2 was performed in a certified biosafety level 3 laboratory. Live SARS-CoV-2 strain was previously isolated from a nasopharyngeal swab of an infected patient from Jiangsu province, China. Briefly, Vero cells were seeded in 24-well plates (200,000 cells/well) and incubated for approximately 16 h until 90-100% confluent. Serial 3-fold dilution of mAbs 1H1 and 9H1 prepared in DMEM containing 2% FBS was then mixed with titrated virus in a 1:1 (vol/vol) ratio to generate a mixture containing 100 focus-forming units (PFU)/ml of viruses, followed by incubation at 37° C. for one h. The complexes of mAb and virus were added to wells of 24-well plates of Vero cell monolayers in duplicate and then incubated at 37° C. for one h. The mixtures were removed, and cells were overlaid with 1% low-melting-point agarose (Promega) in DMEM containing 2% FBS. After incubation at 37° C. for three days, the cells were fixed with 4% formaldehyde and stained with 0.2% crystal violet solution (Sigma). SARS-CoV-2-infected cell foci were visualized through the plaque numbers. The 50% inhibitory concentration (IC50) of mAb was defined as the concentration of antibody (g/mL), resulting in a 50% reduction relative to the total number of plaques counted without antibody.

8. ELISA Assay for ACE2 Receptor Blocking.

Recombinant ACE2 (Kactus Biosystems, Cat. No. ACE-HM501) was coated at 1 μg/ml on an ELISA plate. Antibody was pre-incubated with RBD domain protein diluted at different concentrations for one h at room temperature. Antibody-RBD complexes were then deposited to ACE2-coated ELISA plate for one h at room temperature.

The term “a,” “an,” or “the” cover both the singular and the plural reference unless the context dictates otherwise. The terms “comprising,” “having,” “including,” and “containing” are open-ended terms, which means “including but not limited to,” unless otherwise indicated.

While the disclosure has been illustrated and described in typical embodiments, it is not intended to be limited to the details shown above since various modifications and substitutions can be made without departing from the present disclosure. As such, further modifications and equivalents of the disclosure herein disclosed may occur to persons having ordinary skills in the art using no more than routine experimentation. Such modifications and equivalents of the disclosure herein encompass nuclear acid sequences encoding the amino acid sequences disclosed.

Aspects

Aspect 1. An antibody for recognizing the SARS-CoV-2 S1, comprising:

(a) a V_{H} CDR1 selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and conservative modifications thereof;

(b) a V_{H} CDR2 selected from the group consisting SEQ ID NO: 8, SEQ ID NO: 9, SEQ ID NO: 10, SEQ ID NO: 11, SEQ ID NO: 12, SEQ ID NO: 13, SEQ ID NO: 14, and conservative modifications thereof;

(c) a V_{H} CDR3 selected from the group consisting of SEQ ID NO: 15, SEQ ID NO: 16, SEQ ID NO: 17, SEQ ID NO: 18, SEQ ID NO: 19, SEQ ID NO: 20, SEQ ID NO: 21, and conservative modifications thereof;

(d) a V_{L} CDR1 selected from the group consisting SEQ IDN NO: 22, SEQ ID NO: 23, SEQ ID NO: 24, SEQ ID NO: 25, SEQ ID NO: 26, SEQ ID NO: 27, SEQ ID NO: 28, and conservative modifications thereof;

(e) a V_{L} CDR2 selected from the group consisting of SEQ ID NO: 29, SEQ ID NO: 30, SEQ ID NO: 31, SEQ ID NO: 32, SEQ ID NO: 33, SEQ ID NO: 34, SEQ ID NO: 35, and conservative modifications thereof; and

(f) a V_{L} CDR3 selected from the group consisting of SEQ ID NO: 36, SEQ ID NO: 37, SEQ ID NO: 38, SEQ ID NO: 39, SEQ ID NO: 40, SEQ ID NO: 41, SEQ ID NO: 42, and conservative modifications thereof.

Aspect 2. The antibody of aspect 1, wherein

(a) the V_{H} CDR1 includes the amino acid sequence of SEQ ID NO: 1 or a conservative modification thereof;

(b) the V_{H} CDR2 includes the amino acid sequence of SEQ ID NO: 8 or a conservative modification thereof;

(c) the V_{H} CDR3 includes the amino acid sequence of SEQ ID NO: 15 or a conservative modification thereof;

(d) the V_{L} CDR1 includes the amino acid sequence of SEQ ID NO: 22 or a conservative modification thereof;

(e) the V_{L} CDR2 includes the amino acid sequence of SEQ ID NO: 29 or a conservative modification thereof; and

(f) the V_{L} CDR3 includes the amino acid sequence of SEQ ID NO: 36 or a conservative modification thereof.

Aspect 3. The antibody of aspect 1, wherein

(a) the V_{H} CDR1 includes the amino acid sequence of SEQ ID NO: 2 or a conservative modification thereof;

(b) the V_{H} CDR2 includes the amino acid sequence of SEQ ID NO: 9 or a conservative modification thereof;

(c) the V_{H} CDR3 includes the amino acid sequence of SEQ ID NO: 16 or a conservative modification thereof;

(d) the V_{L} CDR1 includes the amino acid sequence of SEQ ID NO: 23 or a conservative modification thereof;

(e) the V_{L} CDR2 includes the amino acid sequence of SEQ ID NO: 30 or a conservative modification thereof; and

(f) the V_{L} CDR3 includes the amino acid sequence of SEQ ID NO: 37 or a conservative modification thereof.

Aspect 4. The antibody of aspect 1, wherein

(a) the V_{H} CDR1 includes the amino acid sequence of SEQ ID NO: 3 or a conservative modification thereof;

(b) the V_{H} CDR2 includes the amino acid sequence of SEQ ID NO: 10 or a conservative modification thereof;

(c) the V_{H} CDR3 includes the amino acid sequence of SEQ ID NO: 17 or a conservative modification thereof;

(d) the V_{L} CDR1 includes the amino acid sequence of SEQ ID NO: 24 or a conservative modification thereof;

(e) the V_{L} CDR2 includes the amino acid sequence of SEQ ID NO: 31 or a conservative modification thereof; and

(f) the V_{L} CDR3 includes the amino acid sequence of SEQ ID NO: 38 or a conservative modification thereof.

Aspect 5. The antibody of aspect 1, wherein

(a) the V_{H} CDR1 includes the amino acid sequence of SEQ ID NO: 4 or a conservative modification thereof;

(b) the V_{H} CDR2 includes the amino acid sequence of SEQ ID NO: 11 or a conservative modification thereof;

(c) the V_{H} CDR3 includes the amino acid sequence of SEQ ID NO: 18 or a conservative modification thereof;

(d) the V_{L} CDR1 includes the amino acid sequence of SEQ ID NO: 25 or a conservative modification thereof;

(e) the V_{L} CDR2 includes the amino acid sequence of SEQ ID NO: 32 or a conservative modification thereof; and

(f) the V_{L} CDR3 includes the amino acid sequence of SEQ ID NO: 39 or a conservative modification thereof.

Aspect 6. The antibody of aspect 1, wherein

(a) the V_{H} CDR1 includes the amino acid sequence of SEQ ID NO: 5 or a conservative modification thereof;

(b) the V_{H} CDR2 includes the amino acid sequence of SEQ ID NO: 12 or a conservative modification thereof;

(c) the V_{H} CDR3 includes the amino acid sequence of SEQ ID NO: 19 or a conservative modification thereof;

(d) the V_{L} CDR1 includes the amino acid sequence of SEQ ID NO: 26 or a conservative modification thereof;

(e) the V_{L} CDR2 includes the amino acid sequence of SEQ ID NO: 33 or a conservative modification thereof; and

(f) the V_{L} CDR3 includes the amino acid sequence of SEQ ID NO: 40 or a conservative modification thereof.

Aspect 7. The antibody of aspect 1, wherein

(a) the V_{H} CDR1 includes the amino acid sequence of SEQ ID NO: 6 or a conservative modification thereof;

(b) the V_{H} CDR2 includes the amino acid sequence of SEQ ID NO: 13 or a conservative modification thereof;

(c) the V_{H} CDR3 includes the amino acid sequence of SEQ ID NO: 20 or a conservative modification thereof;

(d) the V_{L} CDR1 includes the amino acid sequence of SEQ ID NO: 27 or a conservative modification thereof;

(e) the V_{L} CDR2 includes the amino acid sequence of SEQ ID NO: 34 or a conservative modification thereof; and

(f) the V_{L} CDR3 includes the amino acid sequence of SEQ ID NO: 41 or a conservative modification thereof.

Aspect 8. The antibody of aspect 1, wherein

(a) the V_{H} CDR1 includes the amino acid sequence of SEQ ID NO: 7 or a conservative modification thereof;

(b) the V_{H} CDR2 includes the amino acid sequence of SEQ ID NO: 14 or a conservative modification thereof;

(c) the V_{H} CDR3 includes the amino acid sequence of SEQ ID NO: 21 or a conservative modification thereof;

(d) the V_{L} CDR1 includes the amino acid sequence of SEQ ID NO: 28 or a conservative modification thereof;

(e) the V_{L} CDR2 includes the amino acid sequence of SEQ ID NO: 35 or a conservative modification thereof; and

(f) the V_{L} CDR3 includes the amino acid sequence of SEQ ID NO: 42 or a conservative modification thereof.

Aspect 8. An antibody, comprising:

(a) a V_{H} includes an amino acid sequence selected from the group consisting of SEQ ID NO: 43, SEQ ID NO: 44, SEQ ID NO: 45, SEQ ID NO: 46, SEQ ID NO: 47, SEQ ID NO: 48, SEQ ID NO: 49, and conservative modifications thereof; and

(b) a V_{L} includes an amino acid sequence selected from the group consisting of SEQ ID NO: 50, SEQ ID NO: 51, SEQ ID NO: 52, SEQ ID NO: 53, SEQ ID NO: 54, SEQ ID NO: 55, SEQ ID NO: 56, and conservative modifications thereof;

Aspect 9. The antibody of aspect 8, wherein the V_{H} includes an amino acid sequence of SEQ ID NO: 43, and the V_{L} includes an amino acid sequence of SEQ ID NO:50.
Aspect 10. The antibody of aspect 8, wherein the V_{H} includes an amino acid sequence of SEQ ID NO: 44, and the V_{L} includes an amino acid sequence of SEQ ID NO:51.
Aspect 11. The antibody of aspect 8, wherein the V_{H} includes an amino acid sequence of SEQ ID NO: 45, and the V_{L} includes an amino acid sequence of SEQ ID NO:52.
Aspect 12. The antibody of aspect 8, wherein the V_{H} includes an amino acid sequence of SEQ ID NO: 46, and the V_{L} includes an amino acid sequence of SEQ ID NO:53.
Aspect 13. The antibody of aspect 8, wherein the V_{H} includes an amino acid sequence of SEQ ID NO: 47, and V_{L} includes an amino acid sequence of SEQ ID NO:54.
Aspect 14. The antibody of aspect 8, wherein the V_{H} includes an amino acid sequence of SEQ ID NO: 48, and the V_{L} includes an amino acid sequence of SEQ ID NO: 56.
Aspect 15. The antibody of aspect 8, wherein the V_{H} includes an amino acid sequence of SEQ ID NO: 49, and the V_{L} includes an amino acid sequence of SEQ ID NO: 56.
Aspect 16. An antibody, comprising a Fab fragment including a heavy chain and a light chain,

wherein the heavy chain includes an amino acid sequence of SEQ ID NO: 57, and the light chain includes an amino acid sequence of SEQ ID NO: 64, or

the heavy chain includes an amino acid sequence of SEQ ID NO: 58, and the light chain includes an amino acid sequence of SEQ ID NO: 65, or

the heavy chain includes an amino acid sequence of SEQ ID NO: 59, and the light chain includes an amino acid sequence of SEQ ID NO: 66, or

the heavy chain includes an amino acid sequence of SEQ ID NO: 60, and the light chain includes an amino acid sequence of SEQ ID NO: 67, or

the heavy chain includes an amino acid sequence of SEQ ID NO: 61, and the light chain includes an amino acid sequence of SEQ ID NO: 68, or

the heavy chain includes an amino acid sequence of SEQ ID NO: 62, and the light chain includes an amino acid sequence of SEQ ID NO: 69, and

the heavy chain includes an amino acid sequence of SEQ ID NO: 63, and the light chain includes an amino acid sequence of SEQ ID NO: 70.

Aspect 17. The antibody as in any one of aspects 1-16, wherein the antibody further comprises a covalently or non-covalently attached conjugate.
Aspect 18. The antibody of aspect 17, wherein the conjugate includes an enzyme, a fluorophore, biotin, or streptavidin, or a combination thereof.
Aspect 19. The antibody as in any one of aspects 1-18, wherein the antibody is a humanized or chimeric antibody.
Aspect 20. An ELISA kit for diagnosing SARS-CoV-2 or detecting the SARS-CoV-2 spike S1 protein, comprising the antibody as in any one of aspects 1-19.
Aspect 21. A method for diagnosing SARS-CoV-2 or detecting the SARS-CoV-2 spike S1 protein, comprising:

adding the antibody as in any one of aspects 1-18.

Aspect 22. The method of aspect 21, wherein the method is a direct ELISA.
Aspect 23. The method of aspect 22, wherein the method is a capture ELISA.
Aspect 24. The method of aspect 24, wherein the method is a sandwich ELISA.
Aspect 25. A method for treating a patient infected with SARS-CoV-2, comprising administering the monoclonal antibody (c), (d), (e), (f), or (g) of aspect 1.
Aspect 25. The method of claim 11, wherein the monoclonal antibody (c), (d), (e), (f), or (g) is humanized or chimeric antibody.

We claim:

1. A monoclonal antibody, comprising:
(a) a V_{H} CDR1 having the amino acid sequence of SEQ ID NO: 1, a V_{H} CDR2 having the amino acid sequence of SEQ ID NO: 8, and a V_{H} CDR3 having the amino acid sequence of SEQ ID NO: 15, a V_{L} CDR1 having the amino acid sequence of SEQ ID NO: 22, a V_{L} CDR2 having the amino acid sequence of SEQ ID NO: 29, and a V_{L} CDR3 having the amino acid sequence of SEQ ID NO: 36; or
(b) a V_{H} CDR1 having the amino acid sequence of SEQ ID NO: 2, a V_{H} CDR2 having the amino acid sequence of SEQ ID NO: 9, V_{H} CDR3 having the amino acid sequence of SEQ ID NO: 16, a V_{L} CDR1 having the amino acid sequence of SEQ ID NO: 23, a V_{L} CDR2 having the amino acid sequence of SEQ ID NO: 30, and a V_{L} CDR3 having the amino acid sequence of SEQ ID NO: 37; or
(c) a V_{H} CDR1 having the amino acid sequence of SEQ ID NO: 3, a V_{H} CDR2 having the amino acid sequence of SEQ ID NO: 10, V_{H} CDR3 having the amino acid sequence of SEQ ID NO: 17, a V_{L} CDR1 having the amino acid sequence of SEQ ID NO: 24, a V_{L} CDR2 having the amino acid sequence of SEQ ID NO: 31, and a V_{L} CDR3 having the amino acid sequence of SEQ ID NO: 38; or
(d) a V_{H} CDR1 having the amino acid sequence of SEQ ID NO: 4, a V_{H} CDR2 having the amino acid sequence of SEQ ID NO: 11, a V_{H} CDR3 having the amino acid sequence of SEQ ID NO: 18, a V_{L} CDR1 having the amino acid sequence of SEQ ID NO: 25, a V_{L} CDR2 having the amino acid sequence of SEQ ID NO: 32, and a V_{L} CDR3 having the amino acid sequence of SEQ ID NO: 39; or
(e) a V_{H} CDR1 having the amino acid sequence of SEQ ID NO: 5, a V_{H} CDR2 having the amino acid sequence of SEQ ID NO: 12, a V_{H} CDR3 having the amino acid sequence of SEQ ID NO: 19, a V_{L} CDR1 having the amino acid sequence of SEQ ID NO: 26, a V_{L} CDR2 having the amino acid sequence of SEQ ID NO: 33, and a V_{L} CDR3 having the amino acid sequence of SEQ ID NO: 40; or
(f) a V_{H} CDR1 having the amino acid sequence of SEQ ID NO: 6, a V_{H} CDR2 having the amino acid sequence of SEQ ID NO: 13, a V_{H} CDR3 having the amino acid sequence of SEQ ID NO: 20, a V_{L} CDR1 having the amino acid sequence of SEQ ID NO: 27, a V_{L} CDR2 having the amino acid sequence of SEQ ID NO: 34, and a V_{L} CDR3 having the amino acid sequence of SEQ ID NO: 41;
(g) a V_{H} CDR1 having the amino acid sequence of SEQ ID NO: 7, a V_{H} CDR2 having the amino acid sequence of SEQ ID NO: 14, a V_{H} CDR3 having the amino acid sequence of SEQ ID NO: 21, a V_{L} CDR1 having the amino acid sequence of SEQ ID NO: 28, a V_{L} CDR2 having the amino acid sequence of SEQ ID NO: 35, and a V_{L} CDR3 having the amino acid sequence of SEQ ID NO: 42.

2. The monoclonal antibody of claim 1, comprising:
(a) a V_{H} comprising the amino acid sequence of SEQ ID NO: 43 and a V_{L} comprising the amino acid sequence of SEQ ID NO: 50; or
(b) a V_{H} comprising the amino acid sequence of SEQ ID NO: 44 and a V_{L} comprising the amino acid sequence of SEQ ID NO: 51; or
(c) a V_{H} comprising the amino acid sequence of SEQ ID NO: 45 and a V_{L} comprising the amino acid sequence of SEQ ID NO: 52; or
(d) a V_{H} comprising the amino acid sequence of SEQ ID NO: 46 and a V_{L} comprising the amino acid sequence of SEQ ID NO: 53; or
(e) a V_{H} comprising the amino acid sequence of SEQ ID NO: 47 and a V_{L} comprising the amino acid sequence of SEQ ID NO: 54; or
(f) a V_{H} comprising the amino acid sequence of SEQ ID NO: 48 and a V_{L} comprising the amino acid sequence of SEQ ID NO: 55; or
(g) a V_{H} comprising the amino acid sequence of SEQ ID NO: 49 and a V_{L} comprising the amino acid sequence of SEQ ID NO: 56.

3. The monoclonal antibody of claim 1, comprising:
(a) a heavy chain comprising the amino acid sequence of SEQ ID NO: 57 and a light chain comprising the amino acid sequence of SEQ ID NO: 64; or
(b) a heavy chain comprising the amino acid sequence of SEQ ID NO: 58 and a light chain comprising the amino acid sequence of SEQ ID NO: 65; or
(c) a heavy chain comprising the amino acid sequence of SEQ ID NO: 59 and a light chain comprising the amino acid sequence of SEQ ID NO: 66; or
(d) a heavy chain comprising the amino acid sequence of SEQ ID NO: 60 and a light chain comprising the amino acid sequence of SEQ ID NO: 67; or
(e) a heavy chain comprising the amino acid sequence of SEQ ID NO: 61 and a light chain comprising the amino acid sequence of SEQ ID NO: 68; or
(f) a heavy chain comprising the amino acid sequence of SEQ ID NO: 62 and a light chain comprising the amino acid sequence of SEQ ID NO: 69; or
(g) a heavy chain comprising the amino acid sequence of SEQ ID NO: 63 and a light chain comprising the amino acid sequence of SEQ ID NO: 70.

4. The monoclonal antibody of claim 1, wherein the antibody further comprises a covalently or non-covalently attached conjugate.

5. The monoclonal antibody of claim 4, wherein the conjugate includes an enzyme, a fluorescence protein, a fluorophore, a biotin, or a streptavidin.

6. The monoclonal antibody of claim 4, wherein the enzyme includes HRP.

7. The monoclonal antibody of claim 1, wherein the monoclonal antibody is a humanized or chimeric antibody.

8. The monoclonal antibody of claim 1, wherein the monoclonal antibodies (c), (d), (e), (f), and (g) are capable of treating a patient infected with SARS-CoV-2.

8. A kit for diagnosing SARS-CoV-2 or detecting SARS-CoV-2 spike S1 protein, comprising the monoclonal antibody of claim 1.

9. A method for diagnosing SARS-CoV-2 or detecting SARS-CoV-2 spike S1 protein, comprising:
mixing a sample to be detected with the antibody of claim 1.

10. The method of claim 9, wherein the method is a direct ELISA, and the antibody specifically recognizes the spike S1 protein of SARS-CoV-2.

11. A method for treating a patient infected with SARS-CoV-2, comprising administering the monoclonal antibody (c), (d), (e), (f), or (g) of claim 1.

12. The method of claim 11, wherein the monoclonal antibody (c), (d), (e), (f), or (g) is humanized or chimeric antibody.


SEQUENCE LISTING (70 sequences)

SEQ ID NO: 1 - protein, 10 aa, oryctolagus cuniculus
QSVNMNLLSW

SEQ ID NO: 2 - protein, 11 aa, oryctolagus cuniculus
ESVLSNNRLSW

SEQ ID NO: 3 - protein, 9 aa, oryctolagus cuniculus
ESISNWLAW

SEQ ID NO: 4 - protein, 9 aa, oryctolagus cuniculus
QNIYSNLAW

SEQ ID NO: 5 - protein, 11 aa, oryctolagus cuniculus
QSVYNNDNLAW

SEQ ID NO: 6 - protein, 9 aa, oryctolagus cuniculus
WSIGSNLAW

SEQ ID NO: 7 - protein, 9 aa, oryctolagus cuniculus
EDIYDNLVW

SEQ ID NO: 8 - protein, 12 aa, oryctolagus cuniculus
LIYQASNLASGV

SEQ ID NO: 9 - protein, 12 aa, oryctolagus cuniculus
LIYAASTLASGV

SEQ ID NO: 10 - protein, 12 aa, oryctolagus cuniculus
LIYAAFTLASGV

SEQ ID NO: 11 - protein, 12 aa, oryctolagus cuniculus
LIYDASQLASGV

SEQ ID NO: 12 - protein, 12 aa, oryctolagus cuniculus
LIYLASNLASGV

SEQ ID NO: 13 - protein, 12 aa, oryctolagus cuniculus
LIYQASNLASGV

SEQ ID NO: 14 - protein, 12 aa, oryctolagus cuniculus
LIYDASTLAFGV

SEQ ID NO: 15 - protein, 11 aa, oryctolagus cuniculus
GDMGGWMFPFG

SEQ ID NO: 16 - protein, 13 aa, oryctolagus cuniculus
GAFSGSSDTRAFG

SEQ ID NO: 17 - protein, 13 aa, oryctolagus cuniculus
QTYSSRDVDNVFG

SEQ ID NO: 18 - protein, 13 aa, oryctolagus cuniculus
QGFESSDIFNVFG

SEQ ID NO: 19 - protein, 14 aa, oryctolagus cuniculus
GGYDCSNADCHAFG

SEQ ID NO: 20 - protein, 14 aa, oryctolagus cuniculus
MNYYISSSYTYTFG

SEQ ID NO: 21 - protein, 14 aa, oryctolagus cuniculus
GEFSCSSGDCTAFG

SEQ ID NO: 22 - protein, 9 aa, oryctolagus cuniculus
FSFSSYHMG

SEQ ID NO: 23 - protein, 9 aa, oryctolagus cuniculus
IDIETYWMS

SEQ ID NO: 24 - protein, 10 aa, oryctolagus cuniculus
FSFSSGYDMC

SEQ ID NO: 25 - protein, 9 aa, oryctolagus cuniculus
IDLSSYAMG

SEQ ID NO: 26 - protein, 10 aa, oryctolagus cuniculus
FSFSSAYYMC

SEQ ID NO: 27 - protein, 9 aa, oryctolagus cuniculus
FSLSAYQMI

SEQ ID NO: 28 - protein, 9 aa, oryctolagus cuniculus
FSLSRYAMS

SEQ ID NO: 29 - protein, 19 aa, oryctolagus cuniculus
WIGTLIGIAGNTYYASWAK

SEQ ID NO: 30 - protein, 18 aa, oryctolagus cuniculus
WIAIITSHDHSGYANWAE

SEQ ID NO: 31 - protein, 20 aa, oryctolagus cuniculus
WIACIGTGSSGNIYYASWAK

SEQ ID NO: 32 - protein, 18 aa, oryctolagus cuniculus
YIGIIYISGLTYYASWAK

SEQ ID NO: 33 - protein, 20 aa, oryctolagus cuniculus
WIACIGVDSGGNSYYASWAK

SEQ ID NO: 34 - protein, 18 aa, oryctolagus cuniculus
YIGIMHTGTSAYYANWAK

SEQ ID NO: 35 - protein, 18 aa, oryctolagus cuniculus
WIGIIVDSGHTAYASWAK

SEQ ID NO: 36 - protein, 15 aa, oryctolagus cuniculus
YWCARIVTATFEFWG

SEQ ID NO: 37 - protein, 16 aa, oryctolagus cuniculus
YFCAKDVGHSTYDLWG

SEQ ID NO: 38 - protein, 20 aa, oryctolagus cuniculus
YFCARDDADYAGPDYFNLWG

SEQ ID NO: 39 - protein, 18 aa, oryctolagus cuniculus
YFCARGEYNSHSHYLLWG

SEQ ID NO: 40 - protein, 11 aa, oryctolagus cuniculus
YFCTRSFSLWG

SEQ ID NO: 41 - protein, 21 aa, oryctolagus cuniculus
YFCGRNLNEGFTGAYPFNLWG

SEQ ID NO: 42 - protein, 19 aa, oryctolagus cuniculus
YFCARETGGGAFYVFEFWG

SEQ ID NO: 43 - protein, 114 aa, oryctolagus cuniculus
QSLEESGGRLVTPGTPLTLTCTVSGFSFSSYHMGWVRQAPGEGLEWIGTLIGIAGNTYYASWAKGRFSISKTSTTVDLKMTSPTTEDTATYWCARIVTATFEFWGPGTLVTVSS

SEQ ID NO: 44 - protein, 114 aa, oryctolagus cuniculus
QSVEESGGRLVTPGTSLTLTCTVSGIDIETYWMSWVRQAPGKGLEWIAIITSHDHSGYANWAEGRFTISKTSTTVTLTITDLQPSDTGTYFCAKDVGHSTYDLWGPGTLVTVSS

SEQ ID NO: 45 - protein, 122 aa, oryctolagus cuniculus
QSLEESGGDLVKPGASLTLTCTASGFSFSSGYDMCWVRQAPGKGLEWIACIGTGSSGNIYYASWAKGRFTISKTSSTTVTLQMTSLTAADTATYFCARDDADYAGPDYFNLWGPGTLVTVSS

SEQ ID NO: 46 - protein, 116 aa, oryctolagus cuniculus
QSVEESGGRLVTPGTPLTLTCTVSGIDLSSYAMGWVRQAPGKGLEYIGIIYISGLTYYASWAKGRFTISKTSTTVDLKIPSPTTEDTATYFCARGEYNSHSHYLLWGPGTLVTVSS

SEQ ID NO: 47 - protein, 114 aa, oryctolagus cuniculus
QEQLEESGGDLVKPGASLTLTCTASGFSFSSAYYMCWVRQAPGKGLEWIACIGVDSGGNSYYASWAKGRFTISTTSSTTVTLQMTSLTAADKATYFCTRSFSLWGPGTLVTISS

SEQ ID NO: 48 - protein, 120 aa, oryctolagus cuniculus
QSVEESGGRLVTPGTPLTLTCTVSGFSLSAYQMIWVRQTPGKGLEYIGIMHTGTSAYYANWAKGRFTISKTSSTTVDLKMTSPTTEDTATYFCGRNLNEGFTGAYPFNLWGPGTLVAVSS

SEQ ID NO: 49 - protein, 117 aa, oryctolagus cuniculus
QSVEESGGRLVTPGTPLTLTCTVSGFSLSRYAMSWVRQAPGKGLEWIGIIVDSGHTAYASWAKGRFTISRTSTTVDLKMTSLTTEDTATYFCARETGGGAFYVFEFWGPGTVVTVSS

SEQ ID NO: 50 - protein, 109 aa, oryctolagus cuniculus
AQVLTQTPSPVSAAVGGTVTISCQASQSVNMNLLSWFQQKPGQPPKLLIYQASNLASGVSARFKGSGSGTQFTLTINEIQCDDAATYYCQGDMGGWMFPFGGGTEVVVT

SEQ ID NO: 51 - protein, 112 aa, oryctolagus cuniculus
AAVLTQTPSPVSAAVGGTVTISCQSSESVLSNNRLSWYQQKPGQPPKLLIYAASTLASGVPSRFKGRGSGTQFTLTISDVQCDDAAMYVCAGAFSGSSDTRAFGGGTEVVVK

SEQ ID NO: 52 - protein, 111 aa, oryctolagus cuniculus
ADIVMTQTPASVSEPVGGTVTIKCQASESISNWLAWYQQKPGQPPKLLIYAAFTLASGVPSRFKGSGSGTQFTLTINGVECADAATYYCQQTYSSRDVDNVFGGGTEVVVK

SEQ ID NO: 53 - protein, 110 aa, oryctolagus cuniculus
AYDMTQTPSSVSEPVGGTVSIKCQASQNIYSNLAWYQQKPGQRPKLLIYDASQLASGVPSRFKGSGSGTEYTLTISGVECADAATYYCQQGFESSDIFNVFGGGTEVVVK

SEQ ID NO: 54 - protein, 114 aa, oryctolagus cuniculus
WRKWLTQTASSVSAAVGGTVTISCQASQSVYNNDNLAWFQQRPGQPPKLLIYLASNLASGVPPRFSGSGSGTQFTLTISDVQCDDAATYYCLGGYDCSNADCHAFGGGTEVVVK

SEQ ID NO: 55 - protein, 112 aa, oryctolagus cuniculus
ADVVMTQTPASVEAAVGGTVTIKCQASQSIGSNLAWYQKKPGQPPKLLIYQASNLASGVPSRFKGSGSGTQFTLTISDLECADAATYYCQMNYYISSSYTYTFGGGTEVVVK

SEQ ID NO: 56 - protein, 111 aa, oryctolagus cuniculus
AQVLTQTPSSVSAAVGGTVTINCQASEDIYDNLVWYQQKPGQPPKLLIYDASTLAFGVSSRFRGSGSGTHFTLTMRDVQCDDAATYYCQGEFSCSSGDCTAFGGGTEVVVK

SEQ ID NO: 57 - protein, 209 aa, oryctolagus cuniculus
QSLEESGGRLVTPGTPLTLTCTVSGFSFSSYHMGWVRQAPGEGLEWIGTLIGIAGNTYYASWAKGRFSISKTSTTVDLKMTSPTTEDTATYWCARIVTATFEFWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 58 - protein, 209 aa, oryctolagus cuniculus
QSVEESGGRLVTPGTSLTLTCTVSGIDIETYWMSWVRQAPGKGLEWIAIITSHDHSGYANWAEGRFTISKTSTTVTLTITDLQPSDTGTYFCAKDVGHSTYDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 59 - protein, 217 aa, oryctolagus cuniculus
QSLEESGGDLVKPGASLTLTCTASGFSFSSGYDMCWVRQAPGKGLEWIACIGTGSSGNIYYASWAKGRFTISKTSSTTVTLQMTSLTAADTATYFCARDDADYAGPDYFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 60 - protein, 211 aa, oryctolagus cuniculus
QSVEESGGRLVTPGTPLTLTCTVSGIDLSSYAMGWVRQAPGKGLEYIGIIYISGLTYYASWAKGRFTISKTSTTVDLKIPSPTTEDTATYFCARGEYNSHSHYLLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 61 - protein, 209 aa, oryctolagus cuniculus
QEQLEESGGDLVKPGASLTLTCTASGFSFSSAYYMCWVRQAPGKGLEWIACIGVDSGGNSYYASWAKGRFTISTTSSTTVTLQMTSLTAADKATYFCTRSFSLWGPGTLVTISSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 62 - protein, 215 aa, oryctolagus cuniculus
QSVEESGGRLVTPGTPLTLTCTVSGFSLSAYQMIWVRQTPGKGLEYIGIMHTGTSAYYANWAKGRFTISKTSSTTVDLKMTSPTTEDTATYFCGRNLNEGFTGAYPFNLWGPGTLVAVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 63 - protein, 212 aa, oryctolagus cuniculus
QSVEESGGRLVTPGTPLTLTCTVSGFSLSRYAMSWVRQAPGKGLEWIGIIVDSGHTAYASWAKGRFTISRTSTTVDLKMTSLTTEDTATYFCARETGGGAFYVFEFWGPGTVVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 64 - protein, 213 aa, oryctolagus cuniculus
AQVLTQTPSPVSAAVGGTVTISCQASQSVNMNLLSWFQQKPGQPPKLLIYQASNLASGVSARFKGSGSGTQFTLTINEIQCDDAATYYCQGDMGGWMFPFGGGTEVVVTGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 65 - protein, 216 aa, oryctolagus cuniculus
AAVLTQTPSPVSAAVGGTVTISCQSSESVLSNNRLSWYQQKPGQPPKLLIYAASTLASGVPSRFKGRGSGTQFTLTISDVQCDDAAMYVCAGAFSGSSDTRAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 66 - protein, 215 aa, oryctolagus cuniculus
ADIVMTQTPASVSEPVGGTVTIKCQASESISNWLAWYQQKPGQPPKLLIYAAFTLASGVPSRFKGSGSGTQFTLTINGVECADAATYYCQQTYSSRDVDNVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 67 - protein, 214 aa, oryctolagus cuniculus
AYDMTQTPSSVSEPVGGTVSIKCQASQNIYSNLAWYQQKPGQRPKLLIYDASQLASGVPSRFKGSGSGTEYTLTISGVECADAATYYCQQGFESSDIFNVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 68 - protein, 218 aa, oryctolagus cuniculus
WRKWLTQTASSVSAAVGGTVTISCQASQSVYNNDNLAWFQQRPGQPPKLLIYLASNLASGVPPRFSGSGSGTQFTLTISDVQCDDAATYYCLGGYDCSNADCHAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 69 - protein, 216 aa, oryctolagus cuniculus
ADVVMTQTPASVEAAVGGTVTIKCQASQSIGSNLAWYQKKPGQPPKLLIYQASNLASGVPSRFKGSGSGTQFTLTISDLECADAATYYCQMNYYISSSYTYTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 70 - protein, 215 aa, oryctolagus cuniculus
AQVLTQTPSSVSAAVGGTVTINCQASEDIYDNLVWYQQKPGQPPKLLIYDASTLAFGVSSRFRGSGSGTHFTLTMRDVQCDDAATYYCQGEFSCSSGDCTAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC